(19) United States
(12) Patent Application Publication
(10) Pub. No.: US 2024/0042054 A1
(43) Pub. Date: Feb. 8, 2024

(54) HYDROGEL COMPOSITIONS COMPRISING GASDERMIN D AND AN ESCRT INHIBITOR AND METHODS OF USE THEREOF

(71) Applicant: Wisconsin Alumni Research Foundation, Madison, WI (US)

(72) Inventors: Quanyin Hu, Madison, WI (US); Zhaoting Li, Madison, WI (US)

(21) Appl. No.: 18/354,104

(22) Filed: Jul. 18, 2023

Related U.S. Application Data
(60) Provisional application No. 63/390,444, filed on Jul. 19, 2022.

(51) Int. Cl. A61K 47/69; A61P 35/00; A61K 31/222; A61K 9/51; A61K 39/395

(52) U.S. Cl. CPC A61K 47/6901; A61P 35/00; A61K 31/222; A61K 9/5161; A61K 9/5138; A61K 39/3955; A61K 47/6903

(57) ABSTRACT

Described herein is hydrogel composition, wherein the hydrogel is loaded with bacterial particles including a gasdermin D (GSDMD) protein cage conjugated to a surface thereof, and nanoparticles loaded with an ESCRT inhibitor. The hydrogel can be formulated as an injectable hydrogel for treatment at the site of a primary or metastatic tumor. Alternatively, the hydrogel can be formulated as a thermosensitive hydrogel for implantation at the site of an inoperable cancer.

### CROSS-REFERENCE TO RELATED APPLICATIONS

This application claims priority to U.S. Provisional Application 63/390,444 filed on Jul. 19, 2022, which is incorporated herein by reference in its entirety.

### SEQUENCE LISTING

The Instant Application contains a Sequence Listing which has been submitted electronically in XML format and is hereby incorporated by reference in its entirety. Said XML copy, created on Jul. 12, 2023, is named “Replacement_Seq_List-WIS0070US2” and is 3,686 bytes in size. The Sequence Listing does not go beyond the disclosure in the application as filed.

### FIELD OF THE DISCLOSURE

The present disclosure is related to hydrogel compositions loaded with at least two types of particles, and methods of using the loaded hydrogels to treat primary and inoperable tumors through pyroptosis.

### BACKGROUND

Pyroptosis, a type of programmed cell death mediated by gasdermin proteins, is characterized by the continuous expansion of cells forming large ballooning bubbles until the cell membrane ruptures, resulting in the release of cellular contents and subsequent activation of a strong inflammatory response. As an important innate immune response in the body, pyroptosis plays a crucial role in antagonizing infection and endogenous danger signals. Moreover, the latest research reveals that cytotoxic lymphocytes rely on gasdermin-mediated pyroptosis to kill tumor cells, suggesting that pyroptosis is also closely involved in anti-cancer immune response and rising as a very promising method for cancer treatment. Specifically, granzyme A released from cytotoxic lymphocytes cleaves gasdermin B (GSDMB) to trigger pyroptosis in target tumor cells, while cytotoxic lymphocytes secreted granzyme B or chemotherapy induced activated caspase 3 cleaves gasdermin E (GSDME) to trigger tumor pyroptosis. In addition, it is believed that activated caspase 8 could cleave gasdermin C (GSDMC) to trigger tumor pyroptosis. Moreover, the working mechanism of gasdermin A (GSDMA) in tumor pyroptosis is unclear, but it was found that GSDMA could also trigger tumor pyroptosis and exhibit antitumor efficacy.

What is needed are improved compositions and methods to stimulate pyroptosis.

### BRIEF SUMMARY

In an aspect, a composition comprises a hydrogel, the hydrogel loaded with a) bacterial particles comprising a gasdermin D (GSDMD) protein cage conjugated to a surface thereof, and b) nanoparticles loaded with an ESCRT inhibitor. The hydrogel can be an injectable hydrogel or a thermosensitive hydrogel.

In another aspect, a method of treating a primary or metastatic tumor comprises locally injecting the injectable hydrogel described above at the site of the primary or metastatic tumor.

In a further aspect, a method of treating an inoperable cancer comprises implanting the thermosensitive hydrogel described above at the site of the inoperable cancer.

### BRIEF DESCRIPTION OF THE DRAWINGS

FIGS. 1a-h illustrate the working mechanism and preparation and characterization of the hydrogel-based bacteria protein cage delivery system. 1a shows the GSDMD proteins were crosslinked into protein cages and then conjugated on the surface of attenuated Salmonella typhimurium (designated VNP-GD). The ESCRT inhibitor (a Ca²⁺ chelator BAPTA-AM) was loaded in the dextran nanoparticles (designated EI-NP). Two formulations, an injectable hydrogel and a cell patch, were developed to co-load VNP-GD and EI-NP to treat primary tumors through local administration and inoperable cancer (e.g., advanced ovarian cancer) through implantation, respectively. 1b shows the underlying mechanism of tumor pyroptosis triggered by VNP-GD and further enhanced by EI-NP. Firstly, after the invasion of the VNP bacteria into the tumor, the GSDMD protein would be released upon GSH stimulation, and the abundant flagella on the surface of bacteria could activate the caspase 1 into cleaved caspase 1, which will further cleave the GSDMD protein to the N-terminal GSDMD (GSDMD-NT) that will multimerize and perforate the cell membrane, initiating the pyroptosis. Secondly, during the pyroptosis, the calcium outside the cell can flow into the cell through the formed pore on the membrane which will trigger the ESCRT III-mediated membrane repair thus protecting the tumor cells from pyroptosis through budding and microvesicle formation. Notably, the released ESCRT inhibitor from the dextran nanoparticle could effectively block the calcium influx to inhibit the ESCRT III-mediated membrane repair to enhance the tumor pyroptosis. 1c shows the particle size and transmission electronic microscope (TEM) image (inserted) of the protein cages (scale bar=100 nm). 1d shows representative TEM image of the protein cage-conjugated VNP bacteria (VNP-GD, scale bar=200 nm). 1e shows confocal images of the conjugation of the protein cage (labeled with Rhodamine B) on the surface of the VNP bacteria (labeled with Hoechst), scale bar=10 μm. 1f shows cumulative release of the protein from the bacteria protein cage with or without the trigger of GSH (10 mM). 1g shows particle size and transmission electronic microscope (TEM) image (inserted) of the EI-NP (scale bar=500 nm). 1h shows ESCRT inhibitor release profile from the dextran nanoparticle (EI-NP) at predetermined time points.

FIGS. 2a and 2b illustrate the synthesis route and ¹H NMR characterization of the GSH responsive linker. 2a shows the synthesis route of the GSH responsive linker (R—S). 2b shows the ¹H NMR characterization of the GSH responsive linker (R—S).

FIG. 3 shows the zeta potential of the gasdermin protein cage GD and ESCRT inhibitor loaded nanoparticle EI-NP. Data are shown as mean±s.d. (n=3 biologically independent samples).

FIG. 4 shows representative TEM image of VNP-GD. Scale bar=1 μm.

FIG. 5 shows representative TEM image of VNP. Scale bar=500 nm.

FIG. 6 shows flow cytometry assay of the conjugation efficiency of Rhodamine B-labeled gasdermin D protein cages on the surface of VNP.

FIG. 7 shows flow cytometry assay of the cell uptake of VNP-GD-RhoB in 4T1 cells. The protein cages were labeled with NHS-Rhodamine B before conjugation onto the bacteria. Untreated 4T1 cells served as control.

FIG. 8 shows confocal images of penetration of VNP-GD. The protein cages were labeled with NHS-Rhodamine B before conjugation onto VNP. Scale bar=100 μm.

FIGS. 9a-f show characterization and verification of VNP-GD-induced tumor cell pyroptosis. 9a shows direct observation of the pyroptosis of 4T1 cells under confocal microscope after different treatments with PBS, VNP, GD (GSDMD protein cage), VNP-GD (GSDMD protein cage-conjugated VNP), and VNP-GD+EI-NP (GSDMD protein cage-conjugated VNP+EI-NP) for 24 hours (GSDMD=2 μM, EI=4 μM, VNP=10⁶ CFU mL⁻¹). 4T1 cells were further stained with Annexin V for imaging. Scale bar: 15 μm. 9b and 9c show flow cytometry analysis of cell uptake of SYTOX@ green (9b) and PI (9c) in 4T1 tumor cells after incubation with PBS, VNP, GD (GSDMD protein cage), VNP-GD (GSDMD protein cage-conjugated VNP), and VNP-GD+EI-NP (GSDMD protein cage-conjugated VNP+EI-NP) for 24 hours (GSDMD=2 μM, EI=4 μM, VNP=10⁶ CFU mL⁻¹). 9d shows data analysis of the PI positive cells after different treatments. Data are presented as mean±s.d. (n=3 biologically independent samples) and analyzed with one-way ANOVA followed by Dunnett's multiple comparisons test. VNP-GD+EI-NP vs. VNP-GD: **P=0.0055; VNP-GD+EI-NP vs. GD: ****P<0.0001. 9e and 9f show LDH (9e) and HMGB1 (9f) release after the treatments of PBS, VNP, GD (GSDMD protein cage), VNP-GD (GSDMD protein cage-conjugated VNP), and VNP-GD+EI-NP (GSDMD protein cage-conjugated VNP+EI-NP) for 24 hours (GSDMD=2 μM, EI=4 μM, VNP=10⁶ CFU mL⁻¹). Data are presented as mean±s.d. (n=3 biologically independent samples) and analyzed with one-way ANOVA followed by Dunnett's multiple comparisons test. (e, VNP-GD+EI-NP vs. VNP-GD: **P=0.0068; VNP-GD+EI-NP vs. GD: ***P=0.0001; f, VNP-GD+EI-NP vs. VNP-GD: *P=0.0119; VNP-GD+EI-NP vs. GD: ***P=0.0007).

FIG. 10 shows representative images of B16F10 undergoing pyroptosis after treatment of PBS and VNP-GD+EI-NP for 24 h (GSDMD=2 μM, EI=4 μM, VNP=10⁶ CFU mL⁻¹). Scale bar=20 μm.

FIG. 11 shows data analysis of the cell uptake of SYTOX® green in 4T1 tumor cells after incubation with PBS, VNP, GD (GSDMD protein cage), VNP-GD (GSDMD protein cage-conjugated VNP), and VNP-GD+EI-NP (GSDMD protein cage-conjugated VNP+EI-NP) for 24 hours (GSDMD=2 μM, EI=4 μM, VNP=10⁶ CFU mL⁻¹). Data are presented as mean±s.d. (n=3 biologically independent samples) and analyzed with one-way ANOVA followed by Dunnett's multiple comparisons test. VNP-GD+EI-NP vs. VNP-GD: ***P=0.0003; VNP-GD+EI-NP vs. GD: ****P<0.0001.

FIGS. 12a-e show the working mechanism of GSDMD induced pyroptosis and ESCRT III-mediated cell membrane repair. 12a shows a schematic illustration of the pyroptosis related signaling pathway. 12b shows a Western blot assay of the pyroptosis signaling pathway in 4T1 cells after treatments of PBS, VNP, GD (GSDMD protein cage), VNP-GD (GSDMD protein cage-conjugated VNP), and VNP-GD+EI-NP (GSDMD protein cage-conjugated VNP+EI-NP), GSDMD=2 μM, EI=4 μM, VNP=10⁶ CFU mL⁻¹. 12c shows a schematic illustration of the calcium influx induced ESCRT III-mediated membrane repair during cancer cell pyroptosis. 12d shows a flow cytometry assay of the Ca²⁺ influx detection with Fluo-8 AM in 4T1 cells after treatment with PBS, VNP, GD, VNP-GD, and VNP-GD+EI-NP for 24 hours (GSDMD=2 μM, EI=4 μM, VNP=10⁶ CFU mL⁻¹). 12e shows direct observation of the ESCRT III-mediated 4T1 cancer cell membrane repair during pyroptosis after treatment with VNP-GD and VNP-GD+EI-NP (GSDMD=2 μM, EI=4 μM, VNP=10⁶ CFU mL⁻¹) for 24 hours. The cell membrane was stained with Annexin V. The 4T1 cells were genetically engineered to express mCherry-labeled charged multivesicular body protein 3 (CHMP 3) protein, which is the main component of ESCRT III machinery. Scale bar: 5 μm.

FIG. 13a-k show the anti-tumor efficacy in 4T1 metastatic triple-negative breast cancer models and B16F10 melanoma models. 13a shows a schematic illustration of the establishment and treatment strategy in 4T1 breast tumor model. 13b shows 4T1 tumor growth curves after treatments with PBS, VNP@Gel (hydrogel loaded with VNP), GD/EI-NP@Gel (GSDMD protein cage and EI-NP co-loaded in the hydrogel), VNP-GD@Gel (GSDMD protein cage-conjugated VNP loaded in the hydrogel), VNP-GD/EI-NP@Gel (GSDMD protein cage-conjugated VNP and EI-NP co-loaded in the hydrogel) and VNP-GD/EI-NP@Gel+aPD-1 (GSDMD protein cage-conjugated VNP and EI-NP co-loaded in the hydrogel with systemic injection of aPD-1 antibodies). GSDMD=2 mg/kg, EI=5 mg/kg, VNP=10⁷ CFU per mouse, aPD-1=2.5 mg/kg (three doses on day 0, day 2 and day 4). Data are presented as mean±s.d. (n=6 mice) and analyzed with two-way ANOVA followed by Dunnett's multiple comparisons test. VNP-GD/EI-NP@Gel+aPD1 vs. VNP-GD/EI-NP@Gel: *P=0.0259; VNP-GD/EI-NP@Gel+aPD1 vs. VNP-GD@Gel: ***P=0.0001. 13c shows a survival curve of the 4T1 tumor-bearing mice after different treatments (n=6 mice). Data are analyzed with Log-rank (Mantel-Cox) test. VNP-GD/EI-NP@Gel+aPD-1 vs. VNP-GD/EI-NP@Gel: *P=0.0308. 13d shows a schematic illustration of the establishment and treatment strategy of the breast tumor lung metastasis model. 13e shows a representative lung images and H&E assay after different treatments (n=5 mice, scale bar=2 mm for lung tissue images and 200 μm for H&E staining images). 13f shows the number of surface lung metastatic nodules in different treatment groups. Data are presented as mean±s.d. (n=5 mice) and analyzed with one-way ANOVA followed by Dunnett's multiple comparisons test. VNP-GD/EI-NP@Gel+aPD-1 vs. VNP-GD/EI-NP@Gel: *P=0.0135; VNP-GD/EI-NP@Gel+aPD-1 vs. VNP-GD@Gel: ****P<0.0001. 13g shows a schematic illustration of the establishment and treatment strategy in B16F10 melanoma model. 13h shows B16F10 tumor growth curves after the same treatment regimen described above (n=6 mice). 13i shows a survival curve of the B16F10 tumor-bearing mice after the same treatment regimen described above (n=6 mice). Data are analyzed with Log-rank (Mantel-Cox) test (n=6 mice). VNP-GD/EI-NP@Gel+aPD-1 vs. VNP-GD/EI-NP@Gel: **P=0.0031. 13j shows a schematic illustration of the establishment and treatment strategy of the B16F10 double tumor model. 13k shows distant tumor growth curves after different treatments as described above. Data are presented as mean±s.d. (n=6 mice) and analyzed with two-way ANOVA followed by Dunnett's multiple comparisons test. VNP-GD/EI-NP@Gel+aPD1 vs. VNP-GD/EI-NP@Gel: *P=0.0142; VNP-GD/EI-NP@Gel+aPD1 vs. VNP-GD@Gel: ****P<0.0001.

FIG. 14 shows gel formation of the Pluronic® F-127 solution at room temperature and 37° C.

FIG. 15 shows the number of the bacteria released from hydrogel. 1×10⁷ CFU bacteria were loaded in the hydrogel. Data are shown as mean±s.d. (n=3 biologically independent samples).

FIG. 16 shows representative confocal images of the distribution of Rhodamine B-labeled GSDMD in the tumor tissue after peritumoral injection of GD/EI-NP@Gel and VNP-GD/EI-NP@Gel. Scale bar=100 μm.

FIG. 17 shows body weight changes of the 4T1 breast tumor-bearing mice after treatment with PBS, VNP@Gel (hydrogel loaded with VNP), GD/EI-NP@Gel (GSDMD protein cage and EI-NP co-loaded in the hydrogel), VNP-GD@Gel (GSDMD protein cage-armed VNP loaded in the hydrogel), VNP-GD/EI-NP@Gel (GSDMD protein cage-armed VNP and EI-NP co-loaded in the hydrogel) and VNP-GD/EI-NP@Gel+aPD-1 (GSDMD protein cage-armed VNP and EI-NP co-loaded in the hydrogel with systemic injection of aPD-1 antibodies). GSDMD=2 mg/kg, EI=5 mg/kg, VNP=10⁷ CFU per mouse, aPD-1=2.5 mg/kg (three doses on day 0, day 2 and day 4). Data are presented as mean±s.d. (n=6 mice).

FIG. 18 shows body weight changes of the B16F10 tumor-bearing mice after treatment with PBS, VNP@Gel (hydrogel loaded with VNP), GD/EI-NP@Gel (GSDMD protein cage and EI-NP co-loaded in the hydrogel), VNP-GD@Gel (GSDMD protein cage-armed VNP loaded in the hydrogel), VNP-GD/EI-NP@Gel (GSDMD protein cage-armed VNP and EI-NP co-loaded in the hydrogel) and VNP-GD/EI-NP@Gel+aPD-1 (GSDMD protein cage-armed VNP and EI-NP co-loaded in the hydrogel with systemic injection of aPD-1 antibodies). GSDMD=2 mg/kg, EI=5 mg/kg, VNP=10⁷ CFU per mouse, aPD-1=2.5 mg/kg (three doses on day 0, day 2 and day 4). Data are presented as mean±s.d. (n=6 mice).

FIG. 19 shows H&E assay of different organs including heart, liver, spleen, lung and kidney of C57BL/6 mice after treatments with PBS and VNP-GD/EI-NP@Gel+aPD-1. GSDMD=2 mg/kg, EI=5 mg/kg, VNP=10⁷ CFU per mouse, aPD-1=2.5 mg/kg (three doses on day 0, day 2 and day 4). Scale bar=200 μm.

FIGS. 20a-h show preventing ESCRT-dependent cell membrane repair enhanced pyroptosis and augmented anti-tumor immune response. 20a shows a flow cytometry assay of dendritic cell maturation in the lymph nodes after treatments with PBS, VNP@Gel, GD/EI-NP@Gel, VNP-GD@Gel, VNP-GD/EI-NP@Gel and VNP-GD/EI-NP@Gel+aPD-1. GSDMD=2 mg/kg, EI=5 mg/kg, VNP=10⁷ CFU per mouse, aPD-1=2.5 mg/kg (three doses on day 0, day 2 and day 4), n=4 mice. 20b shows a flow cytometry assay of CD8⁺ T cell infiltration in the tumor tissue after treatments as mentioned above, n=4 mice. 20c shows dendritic cell maturation in the lymph nodes after different treatments. Data are shown as mean±s.d. and analyzed with one way ANOVA followed by Tukey's multiple comparisons test, n=4 mice (VNP-GD@Gel vs. VNP-GD/EI-NP@Gel: ***P=0.0008; VNP-GD@Gel vs. VNP-GD/EI-NP@Gel+aPD-1: ***P=0.0002; VNP-GD/EI-NP@Gel vs. VNP-GD/EI-NP@Gel+aPD-1: **P=0.4116). 20d shows the number of CD8⁺ T cells per mg tumor tissue after different treatments. Data are shown as mean±s.d. and analyzed with one way ANOVA followed by Tukey's multiple comparisons test, n=4 mice (VNP-GD@Gel vs. VNP-GD/EI-NP@Gel: *P=0.0215; VNP-GD@Gel vs. VNP-GD/EI-NP@Gel+aPD-1: ****P<0.0001; VNP-GD/EI-NP@Gel vs. VNP-GD/EI-NP@Gel+aPD-1: **P=0.0042). 20e shows the number of Granzyme B+CD8⁺ T cells per mg tumor tissue after different treatments, n=4 mice (VNP-GD@Gel vs. VNP-GD/EI-NP@Gel: **P=0.0065; VNP-GD@Gel vs. VNP-GD/EI-NP@Gel+aPD-1: ****P<0.0001; VNP-GD/EI-NP@Gel vs. VNP-GD/EI-NP@Gel+aPD-1: **P=0.0028). 20f, 20g, and 20h show HMGB1 (20f), IFNγ (20g) and TNFα (20h) expressions in the tumor tissues after different treatments detected by ELISA. Data are shown as mean±s.d. and analyzed with one way ANOVA followed by Tukey's multiple comparisons test, n=4 mice. (f, VNP-GD@Gel vs. VNP-GD/EI-NP@Gel: **P=0.0080; VNP-GD@Gel vs. VNP-GD/EI-NP@Gel+aPD-1: **P=0.0017; VNP-GD/EI-NP@Gel vs. VNP-GD/EI-NP@Gel+aPD-1: ^{ns}P=0.5319. g, VNP-GD@Gel vs. VNP-GD/EI-NP@Gel: *P=0.0256; VNP-GD@Gel vs. VNP-GD/EI-NP@Gel+aPD-1: ***P=0.0002; VNP-GD/EI-NP@Gel vs. VNP-GD/EI-NP@Gel+aPD-1: *P=0.0127. 20 shows VNP-GD@Gel vs. VNP-GD/EI-NP@Gel: **P=0.0047; VNP-GD@Gel vs. VNP-GD/EI-NP@Gel+aPD-1: ****P<0.0001; VNP-GD/EI-NP@Gel vs. VNP-GD/EI-NP@Gel+aPD-1: **P=0.0053.) 20i shows Luminex@-based quantification of cytokines and chemokines, including IL-1α, IFN-γ, TNF-α, MCP-1, IL-12, IL-1β, IL-6, IL-27, IL-17A, GM-CSF.

FIG. 21 shows the gating strategy for flow cytometry assay of CD8⁺ T cell infiltration in the tumor tissue.

FIG. 22 shows a representative image of lyophilized hydrogel patches loaded with EI-NP.

FIG. 23 shows stability of re-suspended dextran nanoparticle (EI-NP) after lyophilization. Data are shown as mean±s.d. (n=5 biologically independent samples).

FIGS. 24a-d show the anti-tumor efficacy of implantable cell patch in inoperable ovarian cancer. 24a shows a schematic illustration of the implantation of the cell patch for the treatment of inoperable ovarian cancer and the representative image of the lyophilized hydrogel patch loaded with EI-NP. Scale bar: 5 mm. 24b shows representative bioluminescence images of the mice bearing ID8-Luc ovarian tumors after different treatments of PBS, VNP@Gel (hydrogel loaded with VNP), GD/EI-NP@Gel (GSDMD protein cage and EI-NP co-loaded in the hydrogel), VNP-GD@Gel (GSDMD protein cage-conjugated VNP loaded in the hydrogel), VNP-GD/EI-NP@Gel (GSDMD protein cage-conjugated VNP and EI-NP co-loaded in the hydrogel) and VNP-GD/EI-NP@Gel+aPD-1 (GSDMD protein cage-conjugated VNP and EI-NP co-loaded in the hydrogel with three times systemic injection of aPD-1 on day 0, day 2 and day 4) and VNP-GD/EI-NP@Patch+aPD1 (GSDMD protein cage-conjugated VNP and EI-NP loaded in the cell patch with three times systemic injection of aPD-1 on day 0, day 2 and day 4). GSDMD=2 mg/kg, EI=5 mg/kg, VNP=10⁷ CFU per mouse, aPD-1=2.5 mg/kg, n=6 mice. 24C shows a data analysis of the normalized intensity of the bioluminescence signals of the mice with different treatments. Data are presented as mean±s.d. (n=6 mice) and analyzed with two-way ANOVA followed by Dunnett's multiple comparisons test. VNP-GD/EI-NP@Gel+aPD1 vs VNP-GD/EI-NP@Gel: **P=0.0079; VNP-GD/EI-NP@Gel+aPD1 vs. VNP-GD@Gel: ***P=0.0005. 24d shows a survival curve of the ovarian tumor bearing mice after different treatment of PBS, VNP@Gel (hydrogel loaded with VNP), GD/EI-NP@Gel (GSDMD protein cage and EI-NP co-loaded in the hydrogel), VNP-GD@Gel (GSDMD protein cage-conjugated VNP loaded in the hydrogel), VNP-GD/EI-NP@Gel (GSDMD protein cage-conjugated VNP and EI-NP co-loaded in the hydrogel) and VNP-GD/EI-NP@Gel+aPD-1 (GSDMD protein cage-conjugated VNP and EI-NP co-loaded in the hydrogel with three times systemic injection of aPD-1 on day 0, day 2 and day 4) and VNP-GD/EI-NP@Patch+aPD1 (GSDMD protein cage-conjugated VNP and EI-NP loaded in the cell patch with three times systemic injection of aPD-1 on day 0, day 2 and day 4). GSDMD=2 mg/kg, EI=5 mg/kg, VNP=10⁷ CFU per mouse, aPD-1=2.5 mg/kg, n=6 mice. Data are presented as mean±s.d. (n=6 mice) and analyzed by Log-rank (Mantel-Cox) test. VNP-GD/EI-NP@Patch+aPD1 vs. VNP-GD@Gel: *P=0.0204. VNP-GD/EI-NP@Gel+aPD1 vs. VNP-GD@Gel: *P=0.0169.

The above-described and other features will be appreciated and understood by those skilled in the art from the following detailed description, drawings, and appended claims.

### DETAILED DESCRIPTION

Among all gasdermin family proteins, membrane perforin gasdermin D (GSDMD) acts as an effective executor of pyroptosis mainly in immune cells. Notably, there is increasing evidence showing that pyroptosis can inhibit cancer cell proliferation by inducing inflammatory cell death. Leveraging pyroptosis-mediated anti-tumor immune response to overcome the immunosuppression is a novel strategy for cancer immunotherapy.

Despite the encouraging progress of pyroptosis-mediated cancer immunotherapy, there are still emerging limitations restricting its wider application. First, the expression of gasdermins in cancer tissue is suppressed by DNA methylation. For example, methylation of DFNA5 (deafness autosomal dominant 5) gene reduced the GSDME expression in most tumor tissues, thus restricting T cell- or NK cell-mediated cytotoxicity to tumor cells. Furthermore, even if there is the expression of full-length gasdermins in tumor cells, pyroptosis cannot occur when the associated caspase signaling pathway is not activated in the tumor cells. More importantly, calcium influx-triggered assembly of the endosomal sorting complexes required for transport (ESCRT) III system could prevent the cell from programmed cell death and further cell lysis through facilitating damaged cell membrane repair, which significantly dampened the tumor pyroptosis. Therefore, providing sufficient gasdermin intracellular delivery, activating the related caspase signaling pathway and preventing ESCRT mediated cell membrane repair are progressive prerequisites for the realization and enhancement of pyroptosis-mediated cancer immunotherapy.

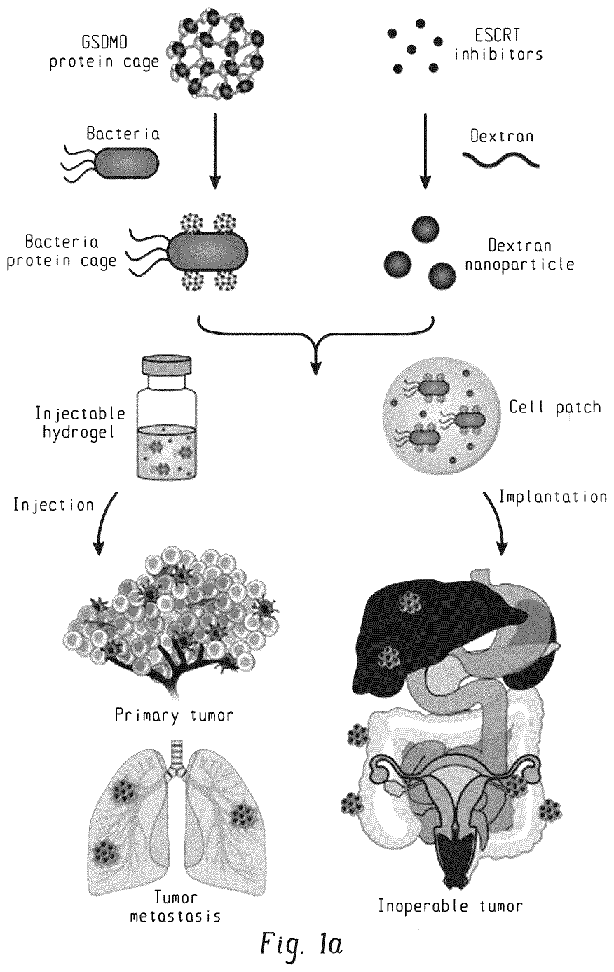

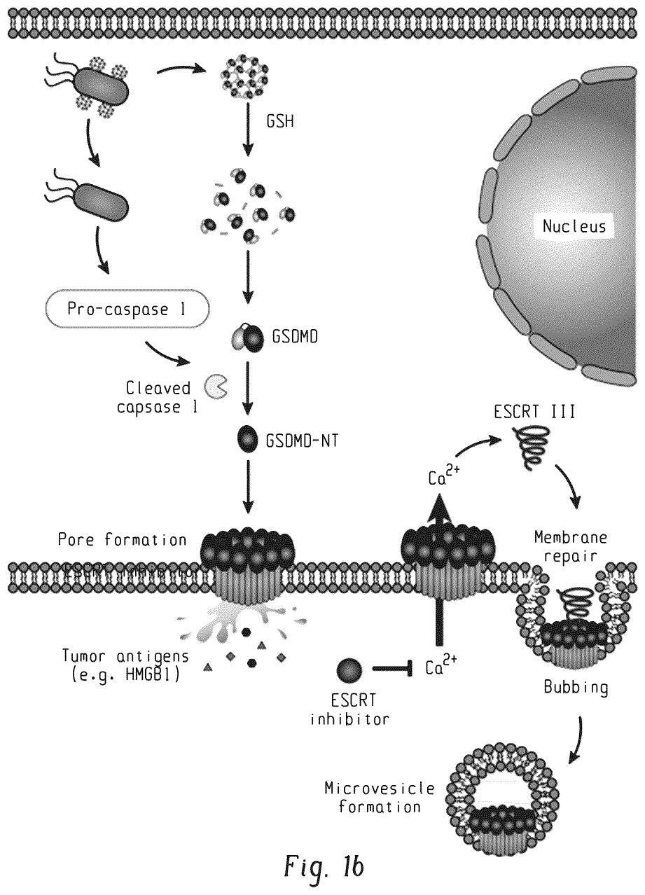

To leverage gasdermin-triggered pyroptosis for anti-tumor immunotherapy, described herein is an intracellular bacterium-attenuated Salmonella typhimurium (VNP) delivery system to shuttle gasdermin D protein to initiate the tumor cell pyroptosis. To facilitate the VNP modification and further intracellular release of GSDMD, GSDMD proteins are assembled into protein nanocages by crosslinking proteins through bifunctional linkers such as glutathione (GSH)-responsive linkers. GSDMD protein cage-conjugated VNP bacteria (designated VNP-GD) could effectively shuttle GSDMD to the cytoplasm of the tumor cells, which will subsequently release GSDMD intracellularly upon the activation of elevated GSH concentration to trigger tumor cell pyroptosis (FIG. 1a). To overcome the plasma membrane repair mediated by ESCRT III machinery, an ESCRT inhibitor (a Ca²⁺ chelator BAPTA-AM) was encapsulated into a biodegradable dextran nanoparticle (designated EI-NP) for sustained release of BAPTA-AM to prevent Ca²⁺ influx-mediated recruitment of ESCRT machinery to the damaged cell membrane (FIG. 1b). Finally, to enable the in vivo treatment, we developed two formulations (an injectable hydrogel and a cell patch) to co-load VNP-GD and EI-NP for primary and metastatic tumors and unresectable tumor treatments (FIG. 1a). We found that in the metastatic breast cancer model, melanoma model and inoperable ovarian cancer model, the tumor cell pyroptosis was triggered by VNP-GD delivery systems, leading to substantial programmed necrotic tumor cells and strong anti-tumor immune response, which were further enhanced by preventing ESCRT-mediated cell membrane repair. Furthermore, this treatment strategy can work synergistically with immune checkpoint blockade therapy to improve the immunotherapy efficacy against multiple immunogenic and non-immunogenic tumor models, paving a promising way to potentially increase the objective response rate of immune checkpoint inhibition in the clinic.

The hydrogel delivery system described herein comprises two types of particles. The first particles include gasdermin D (GSDMD), specifically bacterial particles comprising a GSDMD protein cage conjugated to a surface thereof and the second particles include an endosomal sorting complex required for transport (ESCRT) inhibitor.

The first particles include gasdermin D (GSDMD). As used herein, GSDMD is an executor of pyroptosis in immune cells. While the experiments herein were performed with mouse GSDMSD (SEQ ID NO: 1), the disclosure also include human GSDMSD (SEQ ID NO: 2; accession number NP_001159709), and GSDMSD from other mammalian species. Recombinant GSDMD of SEQ ID NO: 1 is commercially available from MyBioSource.com.

SEQ ID NO: 1
MPSAFEKVVK NVIKEVSGSR GDLIPVDSLR NSTSFRPYCL
LNRKFSSSRF WKPRYSCVNL SIKDILEPSA PEPEPECFGS
FKVSDVVDGN IQGRVMLSGM GEGKISGGAA VSDSSSASMN
VCILRVTQKT WETMQHERHL QQPENKILQQ LRSRGDDLFV
VTEVLQTKEE VQITEVHSQE GSGQFTLPGA LCLKGEGKGH
QSRKKMVTIP AGSILAFRVA QLLIGSKWDI LLVSDEKQRT
FEPSSGDRKA VGQRHHGLNV LAALCSIGKQ LSLLSDGIDE
EELIEAADFQ GLYAEVKACS SELESLEMEL RQQILVNIGK
ILQDQ
SEQ ID NO: 2
MGSAFERVVR RVVQELDHGG EFIPVTSLQS STGFQPYCLV
VRKPSSSWFW KPRYKCVNLS IKDILEPDAA EPDVQRGRSF
HFYDAMDGQI QGSVELAAPG QAKIAGGAAV SDSSSTSMNV
YSLSVDPNTW QTLLHERHLR QPEHKVLQQL RSRGDNVYVV
TEVLQTQKEV EVTRTHKREG SGRFSLPGAT CLQGEGQGHL
SQKKTVTIPS GSTLAFRVAQ LVIDSDLDVL LFPDKKQRTF
QPPATGHKRS TSEGAWPQLP SGLSMMRCLH NFLTDGVPAE
GAFTEDFQGL RAEVETISKE LELLDRELCQ LLLEGLEGVL
RDQLALRALE EALEQGQSLG PVEPLDGPAG AVLECLVLSS
GMLVPELAIP VVYLLGALTM LSETQHKLLA EALESQTLLG
PLELVGSLLE QSAPWQERST MSLPPGLLGN SWGEGAPAWV
LLDECGLELG EDTPHVCWEP QAQGRMCALY ASLALLSGLS
QEPH

In an aspect, the GSDMD is in the form of a protein cage. As used herein, a protein cage is a hollow, typically spherical, typically monodisperse, three-dimensional crosslinked structure which facilitates conjugation of the GSDMD to the surface of a bacteria particle. Exemplary protein cages have diameters of 24.4 to 396 nm, specifically about 130 nm.

The GSDMD protein cages may be prepared by crosslinking GSDMD with a bifunctional linker such as a glutathione (GSH)-responsive linker, which can react with the amino group in the protein and release the protein under GSH trigger. Excess GSH linkers may be removed by ultrafiltration. Furthermore, the protein cages are conjugated on the surface of a bacteria by amide reaction of carboxyl groups in the protein cages and amino groups on the bacteria, and the GSH-responsive linker provides release of the GSDMD protein from the bacteria particle without affecting the activity and structure of the protein when the intracellular GSH causes cleavage of the linker.

Additional bifunctional linkers include reactive oxygen species (ROS)-responsive linkers, pH-responsive linkers, enzyme-responsive linkers, and the like. Exemplary ROS-responsive linkers include thioether-containing polymers, diselenide, thioketal, arylboronic ester, aminoacrylate, oligoproline, peroxalate ester, and mesoporous silicon. Exemplary pH-responsive linkers include pH-sensitive hydrazone linkers, orthoester linkers, weak acid labile linkers, and the like. Exemplary enzyme-responsive linkers include matrix metalloproteinase (MMP)-degradable peptide linkers.

Bacteria such as Salmonella, Clostridium and Bifidobacterium have a natural tropism for solid tumors, and that this tropism may be exploited to facilitate the selective delivery of therapeutic agents to tumor cells. The bacteria particle thus shuttles the GSDMD protein cages to the cytoplasm of the tumor cells. Exemplary bacteria particles include an attenuated Salmonella typhimurium (VNP) delivery system, such as VNP-20009 and Clostridium, Bifidobacterium, Listeria, Streptococcus and Escherichia coli-based delivery systems, such as ECN1917. VNP-20009 (Vion Pharmaceuticals Inc) was created by the chromosomal deletion of two genes, purl (purine biosynthesis) and msbB (LPS biosynthesis) and was attenuated by at least 10,000-fold in mice compared with the parental wild-type strain.

The second particles include an endosomal sorting complex required for transport (ESCRT) inhibitor. Without being held to theory, it is believed that endosomal sorting complexes required for transport (ESCRT) III-mediated cell membrane repair significantly diminishes the tumor cell pyroptosis through forming microvesicles and subsequently removing gasdermin pores from the plasma membrane. The ESCRT inhibitor overcomes the plasma membrane repair mediated by ESCRT III machinery. Exemplary ESCRT inhibitors include the Ca²⁺ chelator BAPTA-AM (1,2-Bis(2-aminophenoxy)ethane-N,N,N′,N′-tetraacetic acid tetrakis(acetoxymethyl ester)), FGI-104 (4-[(7-chloroquinolin-4-yl)amino]-2-(diethylaminomethyl)-6-[4-(hydroxymethyl)-3-methoxyphenyl]phenol), and RNA interference-mediated inhibitors. RNA interference-mediated inhibitors can be used to deplete ESCART proteins such as ESCRT 0, I, II and III.

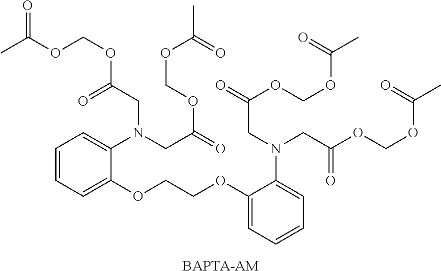

To facilitate delivery of the ESCRT inhibitor, the ESCRT inhibitor is encapsulated in a biodegradable nanoparticle such as a dextran nanoparticle to facilitate sustained release.

As used herein, “biodegradable” materials are those that, when introduced into cells, are broken down by cellular machinery (e.g., enzymatic degradation) or by hydrolysis into components that cells can either reuse or dispose of without significant toxic effects on the cells. In certain aspects, components generated by the breakdown of a biodegradable material do not induce inflammation and/or other adverse effects in vivo. Biodegradable materials may be enzymatically broken down, or broken down by hydrolysis, for example, into their component polymers. Breakdown of biodegradable materials (including, for example, biodegradable polymeric materials) may include hydrolysis of ester bonds, cleavage of urethane linkages, and the like.

Exemplary materials for biodegradable nanoparticles include poly-lactic acid (PLA); poly-D-L-glycolide (PLG); poly-D-L-lactide-co-glycolide (PLGA), poly-alkyl-cyanoacrylate (PCA), poly-ε-caprolactone, gelatin, alginate, chitosan, agarose, polysaccharides, and proteins. Biodegradable nanoparticles can be made by techniques known in the art such as solvent evaporation, spontaneous emulsification, nanoprecipitation, salting out, polymerization, or ionic gelation of hydrophilic polymers, for example.

In an aspect, the biodegradable nanoparticle comprises a polysaccharide. The term “polysaccharide” refers to a polymer of sugars. Polysaccharide nanoparticles described herein may be made of polysaccharides such as dextran, amylose, amylopectin, glycogen, cellulose, arabonixylan, and/or pectin. In certain embodiments, the polysaccharide is dextran. Dextran is a complex, branched glucan (a polysaccharide made of many glucose molecules) composed of chains of varying lengths (from 3-2000 kilodaltons). The straight-chain comprises alpha-1,6-glycosidic linkages between glucose molecules, while branching begins at alpha-1,3 linkages. In some embodiments, dextran nanoparticles are comprised of carboxymethyl dextran.

The polysaccharides that make up the nanoparticles can have a range of molecular weights such as 1 kDa to 1 million kDa (e.g., 1-10 kDa, 10-100 kDa, 100-1000 kDa, or 1000-1,000,000 kDa). The polysaccharide nanoparticles can have an average diameter in a range of 1 nm-500 nm (e.g., 1-10 nm, 10-25 nm, 25-50 nm, 50-100 nm, or 100-500 nm). The polysaccharide nanoparticles may be relatively monodisperse (e.g., diameters of particles all within a range of 10 nm or less of each other) or more polydisperse.

The hydrogel of the delivery system is a substance formed when an organic polymer (natural or synthetic) is cross-linked via covalent, ionic, or hydrogen bonds to create a three-dimensional open-lattice structure that entraps water molecules to form a gel. “Biocompatible hydrogel refers” to a hydrogel that is not toxic to living cells.

Examples of materials that can be used to form a biocompatible hydrogel include polysaccharides such as alginate, polyphosphazines, poly(acrylic acids), poly(methacrylic acids), poly(alkylene oxides), poly(vinyl acetate), polyvinylpyrrolidone (PVP), and copolymers and blends. Additional materials for forming hydrogels include agarose, alginic acid, chitosan, dextran, dextran sulfate, heparan, heparan sulfate, cellulose sulphate, carrageenan, gellan gum, xanthan gum, guar gum, chondroitin sulfate, hyaluronic acid, collagen, gelatin, hydroxyethyl starch and poly(N-isopropyl acrylamide). Combinations of the foregoing materials may be employed.

In an aspect, the hydrogel is a thermosensitive hydrogel. Exemplary materials to form thermosensitive hydrogels include polyoxyethylene-polyoxypropylene (PEO-PPO) block copolymers such as Pluronic® F127 and F108, which are PEO-PPO block copolymers with molecular weights of 12,600 and 14,600, respectively. Each of these compounds is available from BASF (Mount Olive, N.J.). Pluronic® F108 at can form a thermosensitive hydrogel at a concentration of 20-28% in phosphate buffered saline (PBS). Pluronic® F127 at a 20-35% concentration in PBS also forms thermosensitive hydrogels. When the hydrogel is a thermosensitive hydrogel, the composition can be in the form of a hydrogel patch. Advantageously, hydrogel patches can be implanted or placed adjacent to a solid tumor to stimulate pyroptosis and treat cancer.

In another aspect, the hydrogel is an injectable hydrogel. Injectable hydrogels can include polysaccharides such as heparan, heparan sulfate, chitosan, hyaluronic acid, dextran, alginic acid and hydroxyethyl starch. A crosslinker such as a reactive polyethylene glycol crosslinker can be used to form the hydrogel. An exemplary injectable hydrogel is a hyaluronic acid hydrogel, formed by adding Extralink®-Lite (polyethylene glycol diacrylate (PEGDA)) was added to a Glycosil® (thiol-modified hyaluronan). Injectable hydrogels are locally injectable to release therapeutic agents at the site of a solid tumor.

In an aspect, the hydrogel composition further comprises an immune checkpoint inhibitor. When the hydrogel comprising first and second particles is combined with immune checkpoint inhibitors, the tumor immunotherapy efficacy has been substantially augmented, effectively inhibiting tumor growth and prolonging the survival of tumor-bearing mice. Exemplary immune checkpoint inhibitors include peptides with high affinity for an immune checkpoint receptor such as PD-L1, PD-1, OX40, TIGIT, CTLA-4, CD137 (4-1BB), CD28, and CD27.

In an aspect, a method of treating a primary tumor comprises locally injecting the injectable hydrogel described herein at the site of the primary or metastatic tumor. In an aspect, the primary tumor is a metastatic breast cancer tumor, a melanoma, sarcoma, prostate cancer, cervical cancer, and the like. The 4T1 tumor model with lung metastasis is an established mouse model for the lung metastasis of 4T1 breast cancer. The B16F10 tumor model is a B16F10 tumor model is a murine melanoma cell line. In an aspect, the composition further comprises an immune checkpoint inhibitor, such as a peptide with high affinity for an immune checkpoint receptor, wherein the immune checkpoint receptor comprises PD-L1, PD-1, OX40, TIGIT, CTLA-4, CD137 (4-1BB), CD28, and CD27.

In another aspect, a method of treating an inoperable cancer comprises implanting the thermosensitive hydrogel described herein at the site of the inoperable cancer. Exemplary inoperable cancers include inoperable ovarian cancer, lung cancer, pancreatic cancer, liver cancer, and colorectal cancer. The ID8-Luc tumor model is a mouse model for orthotopic ovarian cancer. In an aspect, the composition further comprises an immune checkpoint inhibitor, such as a peptide with high affinity for an immune checkpoint receptor, wherein the immune checkpoint receptor comprises PD-L1, PD-1, OX40, TIGIT, CTLA-4, CD137 (4-1BB), CD28, and CD27.

The invention is further illustrated by the following non-limiting examples.

### EXAMPLES

### Methods

Cells and antibodies: The murine 4T1, B16F10, ID8 cell lines and VNP20009 were purchased from ATCC. Luciferase-expressed B16F10 and 4T1 were obtained from Imanis Life Sciences Inc. Luciferase-expressed ID8 cells were provided by Dr. Paula Hammond's lab at MIT. Cells were cultured in the CO₂ incubator (Fisher) at 37° C. with 5% CO₂ and 90% relative humidity. The antibodies used in this study were summarized as follows (company, clone, category number): GoInVivo™ Purified anti-mouse CD279 (PD-1) (BioLegend, RMP1-14, 114114), Fluorescein isothiocyanate (FITC)-anti-mouse CD45 (BioLegend, 30-F11, 103108), APC-anti-mouse CD3 (BioLegend, 17A2, 100236), FITC-anti-mouse CD4 (BioLegend, GK1.5, 100406), PE-anti-mouse CD8a (BioLegend, 53-6.7, 100708), FITC-anti-mouse IFNγ (BioLegend, XMG1.2, 505806), PerCP/Cy5.5-anti-human/mouse Granzyme B (BioLegend, QA16A02, 372212), PE-anti-mouse CD45 (BioLegend, 30-F11, 103106), Alexa Fluor® 594 anti-mouse CD8a (BioLegend, 53-6.7, 100758), FITC-anti-mouse CD11c (BioLegend, N418, 117306), PE-anti-mouse CD80 (BioLegend, 16-10A1, 104708), APC-anti-mouse CD86 (BioLegend, GL-1, 105012), Precision Count Beads (BioLegend, 424902). All antibody dilutions were performed following the manufacture's guidance (diluted by approximately 200 times for use).

Synthesis of the GSH responsive linker: 2-hydroxyethyl disulphide (R, 200 mg, 1.30 mmol) was dissolved in the anhydrous acetonitrile (ACN, 12 mL). N, N′-disuccinimidyl carbonate (DSC, 1.33 g, 5.19 mmol) and Et₃N (1.05 ml, 7.79 mmol) were added. The mixture was stirred for 8 hours at room temperature under nitrogen protection, followed by the removal of the solvent under vacuum. The crude product was then dissolved in CH₂Cl₂ (20 mL). The solution was washed with saturated NaHCO₃ solution, saturated NH₄Cl solution, brine in sequence, and dried over anhydrous Na₂SO₄. The solvent in the organic phase was evaporated, and the solid that remained was recrystallized with ethyl acetate (20 ml). The resulting white solid was dried under vacuum (RS, 380 mg, yield 65%). ¹H-NMR (CDCl₃, 300 MHz): δ (ppm) 4.60 (t, 4H), 3.07 (t, 4H), 2.86 (s, 8H). ESI (m/z): calcd for C₁₄H₁₆N₂O₁₀S₂, 436.4 [M]; found, 459.0 [M+Na]+.

Synthesis, preparation, and characterization of ESCRT inhibitor-loaded dextran nanoparticles: Dextran (1.0 g, Mn approximately 9-11 kDa) dissolved in 10 ml was added to a flame-dried round-bottom flask, then pyridinium P-toluenesulfonate (PPTS, 15.6 mg, 0.062 mmol) and 2-ethoxypropene (4.16 mL, 37 mmol) were added during stirring. The reaction was stirred at room temperature for 30 min and quenched by adding 1 mL of triethylamine. The precipitated mixture was washed three times in basic water (pH approximately 8) to prevent undesired degradation, centrifugated (8000 rpm, 15 min), and lyophilized to obtain the final white solid powder (m-dextran). To prepare the ESCRT inhibitor BAPTA-AM-loaded dextran nanoparticle, 10 mg m-dextran and 0.5 mg BAPTA-AM were dissolved in 2 mL dichloromethane (DCM). Then, 4 mL 3% poly (vinyl alcohol) (PVA) solution was added, and sonication (2 min in total, 2s on, 2s off, 40% power, ice bath) was performed. Next, the mixture was dispersed in 20 ml 0.3% PVA solution in the beaker and stirred for 1 hour. The emulsion was centrifuged at 14,000 rpm for 35 min to collect the dextran nanoparticles (EI-NP). The supernatant was removed, and EI-NP was dispersed in 1 ml PBS. The particle size and zeta potential were measured with the Malvern Zetasizer instrument. The morphology of EI-NP was observed under Transmission Electron Microscope (TEM) imaging. To investigate the release profile of the ESCRT inhibitor, the EI-NP suspended in 2 ml PBS with 0.1% Tween 80 was loaded in a 20,000 MWCO dialysis cassette (Thermo scientific) for the analysis of drug release by high-performance liquid chromatography (HPLC) at different time points.

Preparation and characterization of the protein cage-conjugated bacteria: To prepare the protein cages, the GSDMD proteins (MyBioSource, 0.0143 μmol) were dissolved in the PBS solution (132 μl, PH 7.4), then the GSH responsive linkers (0.214 μmol) dissolved in the DMSO (9.35 μl) were added and stirred at room temperature for 35 min to form the protein cage. The particle size and zeta potential of the protein cage were measured by the Malvern Zetasizer instrument. The morphology of the protein cages was observed under TEM imaging. Next, for the conjugation of protein cages on the surface of bacteria, the obtained protein cages and EDC/NHS (0.214 μmol) were added into the VNP20009 suspension (10⁸ CFU-ml⁻¹) and incubated for one hour in a 37° C. shaker incubator at 250 rpm. The reaction mixture was centrifuged (4,000 rpm, 5 min) to obtain the protein cage-conjugated bacteria (VNP-GD). The morphology of VNP-GD was observed under TEM imaging.

To verify the successful conjugation of the protein cage on the surface of the bacteria, the protein was labeled with Rhodamine B and formed into protein cages before being conjugated onto the bacteria. Centrifugation (4,000 rpm) was performed to remove the unconjugated protein in the supernatant. Then the bacteria were labeled with Hoechst 33342 for 15 min at room temperature and washed with PBS three times before confocal imaging. The release of proteins from the protein cage-conjugated bacteria with or without the trigger of GSH (10 mM) was further characterized by measuring the protein concentration in the supernatant after centrifugation (4,000 rpm) at different time points.

Characterization and verification of VNP-GD induced tumor cell pyroptosis: 2×10⁵ 4T1 cells were seeded into the confocal dishes, incubated overnight, and then treated with PBS, VNP, GD (GSDMD protein cage), VNP-GD (GSDMD protein cage-conjugated VNP), and VNP-GD+EI-NP (GSDMD protein cage-conjugated VNP+EI-NP) for 24 hours (GSDMD=2 μM, EI=4 μM, VNP=10⁶ CFU mL⁻¹). After washing with PBS, the morphology of cells was observed under a confocal microscope. In addition, the Annexin V (staining at room temperature for 15 min) was used to label the cell membrane after pyroptosis. To further verify pyroptosis-mediated pore formation, 2×10⁵ 4T1 cells were seeded into the 24-well plate, incubated overnight, and then treated with PBS, VNP, GD, VNP-GD, and VNP-GD+EI-NP for 24 hours (GSDMD=2 μM, EI=4 μM, VNP=10⁶ CFU mL⁻¹). SYTOX@ green and PI staining were performed respectively, and the cell uptake of the SYTOX@ green and PI were detected by the flow cytometry. Moreover, the LDH release was detected by the Invitrogen™ CyQUANT™ LDH Cytotoxicity Assay Kit according to the instruction and operation manual. HMGB1 expression was detected by the HMGB1 ELISA Kit accordingly.

Western blot assay of the pyroptosis related signaling pathway: 5×10⁵ 4T1 cells were seeded into 6-well plate, incubated overnight, and then treated with PBS, VNP, GD, VNP-GD, and VNP-G+EI-NP for 24 hours (GSDMD=2 μM, EI=4 μM, VNP=10⁶ CFU mL⁻¹). After washing with PBS, the cells were digested with trypsin and collected into 1.5 ml tubes. Centrifugation (1,000 rpm, 4 min) was performed, and 100 μl Pierce™ RIPA buffer with protease inhibitor cocktail was added to each tube for 1-hour cell lysis with vortex every 10 min. Then, the lysed cells were centrifuged under 14,000 rpm for 30 min. The supernatant was collected, and BCA analysis was performed to determine the protein concentration. The protein samples added with protein loading buffer were boiled for 15 min at 100° C. water bath and stored at −20° C. for further use.

To perform the western blot assay, the 1× running buffer was prepared and gel plate was placed into the gel holder. Then the running buffer was added, and the protein marker and various samples were loaded accordingly. SDS-PAGE was performed at 120 V until the bromophenol blue indicator ran to the bottom. The proteins in the gels were further transferred onto a PVDF membrane (350 mA, 85 min). After incubation with 5% skim milk for 2 hours at room temperature, the PVDF membranes were incubated with the primary rabbit antibodies (anti-Gasdermin D, anti-Cleaved Caspase 1, anti-Cleaved Gasdermin D, anti-HMGB1 and anti-β actin, respectively) at 4° C. overnight. The membranes were then washed with TBST buffer three times (each time 10 min) and incubated with the secondary antibody (Anti-rabbit IgG, HRP-linked) at room temperature for 1 hour. After washing with TBST another three times, Western Blot Substrates were added to the membrane, and western blot images were taken by the iBright™ Imaging Systems.

Flow cytometry assay of calcium influx and the confocal imaging of ESCRT III-mediated cell membrane repair during pyroptosis: For the flow cytometry assay of calcium influx, 2×10⁵ 4T1 cells were seeded into the 24-well plates, incubated overnight, and then treated with PBS, VNP, GD, VNP-GD, and VNP-GD+EI-NP for 24 hours (GSDMD=2 μM, EI=4 μM, VNP=10⁶ CFU mL⁻¹). Cells were washed twice with PBS and incubated with 4 μM Fluo-8 for 45 min in the dark. Then cells were washed twice with PBS and digested with trypsin, and the fluorescence intensity of Fluo-8 was detected by the flow cytometry. For the confocal imaging of ESCRT III-mediated cell membrane repair, first, the plasmid containing the CHMP3-mCherry sequence was constructed by Addgene and extracted with the Monarch@ Plasmid Miniprep Kit according to the manufacture instructions. The plasmid concentration was measured with a microplate reader, and the plasmids were stored at −20° C. for further use. To obtain 4T1 cells expressing CHMP3-mCherry, 2×10⁵ 4T1 cells were seeded into the 24-well plates and incubated overnight. Then, 2.5 μg CHMP3-Mcherry DNA plasmid and P 3000™ Regent were mixed in 125 μl DEME medium without FBS in a 1.5 ml tube (A). Meantime, 7.5 μl Lipofectamine™ 3000 was dissolved in 125 μl DEME medium without FBS in a 1.5 ml tube (B). Then the mixture in tube A was added to tube B and mixed well by pipetting and incubated for 10 min at room temperature. After washing with PBS, 600 μl DEME medium (with 10% FBS) was added to the 4T1 cell-containing confocal dishes. The final mixture in tube B was added slowly and evenly into the confocal dishes containing 4T1 cells. After 36 hours, the expression of CHMP3-mCherry was verified by the confocal imaging. Then 4T1 cells expressing the CHMP3-mCherry were further treated with VNP-GD and VNP-GD+EI-NP for 24 hours (GSDMD=2 μM, EI=4 μM, VNP=10⁶ CFU mL⁻¹). After washing with PBS, the cell membrane was labeled with Annexin V in binding buffer for 15 min under room temperature, and the confocal imaging was performed to observe the ESCRT III-mediated cell membrane repair.

Hydrogel preparation and bacteria release from the hydrogel: To prepare the Pluronic® F127 thermosensitive hydrogel, 2 g Pluronic® F127 was dissolved into 10 ml PBS solution under room temperature to form the 20% hydrogel solution. The gelation time of the 20% Pluronic® F127 hydrogel was measured by incubating the hydrogel at 37° C. incubator, which formed into gel in 60-70s. The injectable Pluronic® F127 hydrogel delivery system was used for the treatment of 4T1 breast cancer model and B16F10 melanoma tumor model. To prepare the hyaluronic acid hydrogel, the Extralink®-Lite (PEGDA) was added to a mixture of Glycosil® (thiol-modified hyaluronan loaded with the bacteria delivery system) at 1:4 ratio, and it took about 30 min for the gelation. Then the bacteria release assay was performed after loading 10⁷ CFU bacteria into the hydrogel. The hydrogel loaded with bacteria was loaded in a cell strainer placed on a 6-well plate, and then the 6-well plate was submerged with PBS. At different time points, 100 μl of PBS solution in the plate was collected, diluted, and spread on LB medium plate with bacterial spreader. The number of released bacteria was calculated by counting the number of the formed clones accordingly. For the ID8 ovarian cancer treatment, the prepared EI-NP was loaded into hyaluronic acid hydrogel and lyophilization was performed to obtain the off the shelf hydrogel patch. VNP-GD was loaded into the hydrogel patch to form the final therapeutic cell patch before implantation. As for the stability assay, the particle size of EI-NP after lyophilization was measured at predetermined time points with the Malvern Zetasizer instrument.

In vivo anti-tumor efficacy: The BALB/c (Female, aged 6-8 weeks) and C57BL/6 mice (Male, aged 6-8 weeks) were purchased from Jackson laboratory. The animal study protocol was approved by the Institutional Animal Care and Use Committee (IACUC) at the University of Wisconsin-Madison. Mice were euthanized at humane endpoints if any of the following criteria were met: (i) weight loss or gain of >20%, (ii) moribund, (iii) severe abdominal swelling, (iv) jaundice, or (v) tumor volume >2000 mm³. To verify the anti-tumor efficacy of our treatment strategies, we first established the 4T1 breast cancer model by implanting 4T1 cells in the breast pad of the BALB/c mice. Seven days later, different formulations were loaded into the Pluronic® F127 hydrogel for peritumoral injection, including PBS, VNP@Gel (hydrogel loaded with VNP), GD/EI-NP@Gel (GSDMD protein cage and EI-NP co-loaded in the hydrogel), VNP-GD@Gel (GSDMD protein cage-conjugated VNP loaded in the hydrogel), VNP-GD/EI-NP@Gel (GSDMD protein cage-conjugated VNP and EI-NP co-loaded in the hydrogel) and VNP-GD/EI-NP@Gel+aPD-1 (GSDMD protein cage-conjugated VNP and EI-NP co-loaded in the hydrogel with systemic injection of aPD-1 antibodies). GSDMD=2 mg/kg, EI=5 mg/kg, VNP=10⁷ CFU per mouse, aPD-1=2.5 mg/kg (three doses on day 0, day 2 and day 4). The tumor volume was measured and calculated based on the equation: length×width²×0.5. The survival of the mice was monitored accordingly. Next, a breast cancer lung metastasis model was established to evaluate the anti-metastasis effect of the hydrogel-based delivery systems. Briefly, 4T1 cells were injected into the breast pad of BALB/c mice on day −7, different treatments were administered by peritumoral injection after 7 days, including PBS, VNP@Gel, GD/EI-NP@Gel, VNP-GD@Gel, VNP-GD/EI-NP@Gel and VNP-GD/EI-NP@Gel+aPD-1. GSDMD=2 mg/kg, EI=5 mg/kg, VNP=10⁷ CFU per mouse, aPD-1=2.5 mg/kg (three doses on day 0, day 2 and day 4). Then, on day 7, 2×10⁵ 4T1 cells were administered to the mice in different treatment groups by i.v. injection through tail vein. On day 21, the mice were euthanized, and the lungs were collected for further analysis. The lungs were washed with saline and fixed with Bouin's solution for 6 hours, and pictures were taken to observe the surface lung metastasis nodules. Furthermore, H&E assays were performed to observe the 4T1 tumor lung metastasis.

To further verify the anti-tumor efficacy of the hydrogel-based delivery systems, a melanoma tumor model was established by injecting B16F10 cells into the right flank of C57BL/6 mice on day −7. Seven days later, different formulations were loaded into the Pluronic® F127 hydrogel for peritumoral injection, including PBS, VNP@Gel, GD/EI-NP@Gel, VNP-GD@Gel, VNP-GD/EI-NP@Gel and VNP-GD/EI-NP@Gel+aPD-1. GSDMD=2 mg/kg, EI=5 mg/kg, VNP=10⁷ CFU per mouse, aPD-1=2.5 mg/kg (three doses on day 0, day 2 and day 4). The tumor volume was measured and calculated using the equation: length×width²×0.5. The survival of the mice was monitored accordingly. To verify if the local treatment strategy could activate the systemic immunity to inhibit the growth of the distant tumor, a double B16F10 tumor model was established. Briefly, on day −7, the primary B16F10 tumor was established by injecting the B16F10 cells into the right flank of the C57BL/6 mice. Six days later, the second tumor was established by injecting B16F10 cells on the left flank of the C57BL/6 mice. The tumor volume of the second tumor was monitored according to the equation: length×width²×0.5.

To extend our developed technology for treating inoperable tumors, an advanced ovarian tumor model was established. Briefly, 1×10⁷ ID8-Luc ovarian tumor cells were injected intraperitoneally into six-week-old female C57BL/6 mice. One week later, the establishment of the ovarian tumor model was verified by the IVIS imaging system. D-luciferin potassium salt was dissolved into PBS and intraperitoneally injected into the mice (100 μl, 3 mg D-luciferin potassium salt per mouse). Five minutes after the injection, bioluminescence imaging was performed to record the distribution and growth of the ID8-Luc cells in the enterocoelia. After the establishment of the inoperable ovarian tumor model, surgery was performed to open the abdominal cavity of the mice, and different hydrogel-based delivery systems including PBS, VNP@Gel, GD/EI-NP@Gel, VNP-GD@Gel, VNP-GD/EI-NP@Gel, VNP-GD/EI-NP@Gel+aPD-1 and VNP-GD/EI-NP@Patch+aPD1 (GSDMD protein cage-conjugated VNP and EI-NP loaded in the cell patch with three times systemic injection of aPD-1) were implanted accordingly. GSDMD=2 mg/kg, EI=5 mg/kg, VNP=10⁷ CFU per mouse, aPD-1=2.5 mg/kg (three doses on day 0, day 2 and day 4). IVIS imaging was performed at predetermined time points to monitor the tumor growth.

In vivo immune activation: To verify the immune activation of our treatment strategies, we established the 4T1 breast cancer model by implanting 4T1 cells in the breast pad of the BALB/c mice. Seven days later, different formulations were loaded into the Pluronic® F127 hydrogel for peritumoral injection, including PBS, VNP@Gel, GD/EI-NP@Gel, VNP-GD@Gel, VNP-GD/EI-NP@Gel and VNP-GD/EI-NP@Gel+aPD-1. GSDMD=2 mg/kg, EI=5 mg/kg, VNP=10⁷ CFU per mouse, aPD-1=2.5 mg/kg (three doses on day 0, day 2 and day 4). Then one week later, the tumors and lymph nodes were harvested, weighed, washed with PBS, cut into small pieces, and digested with DEME medium containing 0.5 mg/ml collagenase for 1 hour at a 37° C. incubator. After the digested tumor tissues were mechanically disrupted, they were filtered through a 40 μm cell strainer. For the analysis of lymph nodes, the cell suspension was stained with anti-mouse CD11c, anti-mouse CD80, and anti-mouse CD86. For the analysis of tumor tissue, the cell suspension was stained with anti-mouse CD3, anti-mouse CD4, anti-mouse CD8a, and anti-mouse Granzyme B antibodies and analyzed using flow cytometry with Attune™ NxT Flow Cytometer software (All these antibodies were diluted by ˜200 times). The cytokines expressions in the tumor tissue after different treatments were analyzed by the LEGENDplex™ Multi-Analyte Flow Assay Kit, and IFNγ and TNFα ELISA kits according to the manufacture's guidance.

Statistics: All the results are shown as mean±s.d. The GraphPad Prism software was used to perform statistical analysis, and analysis of variance (ANOVA) was used to compare multiple groups (>two groups) statistically. Log-rank test was performed for the statistical analysis of the survival study. A P value lower than 0.05 (*P<0.05) was considered the threshold for statistical significance among control groups and experimental groups.

### Example 1: Preparation and Characterization of the Hydrogel-Based Bacteria Protein Cage Delivery System

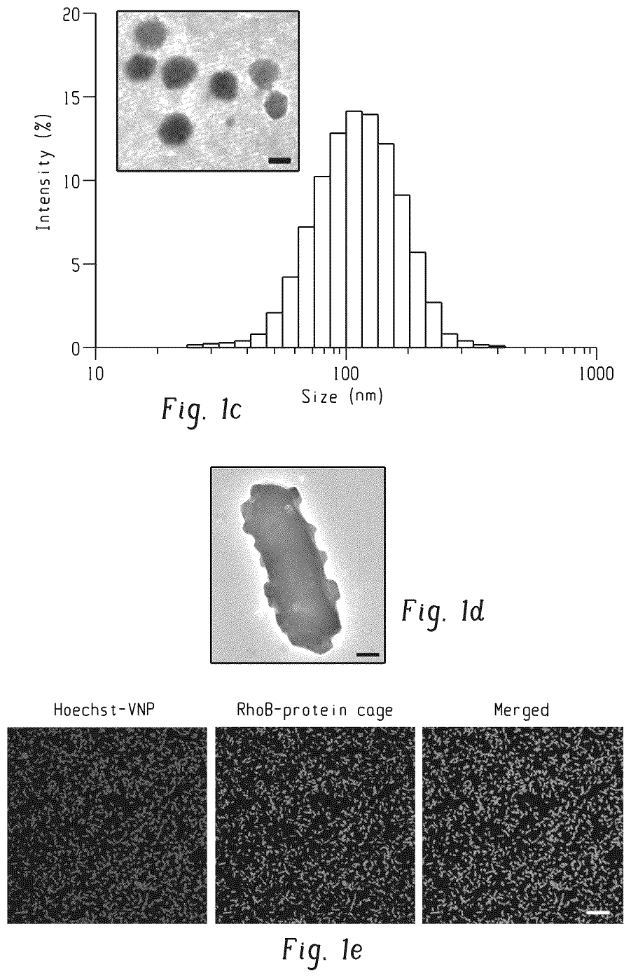

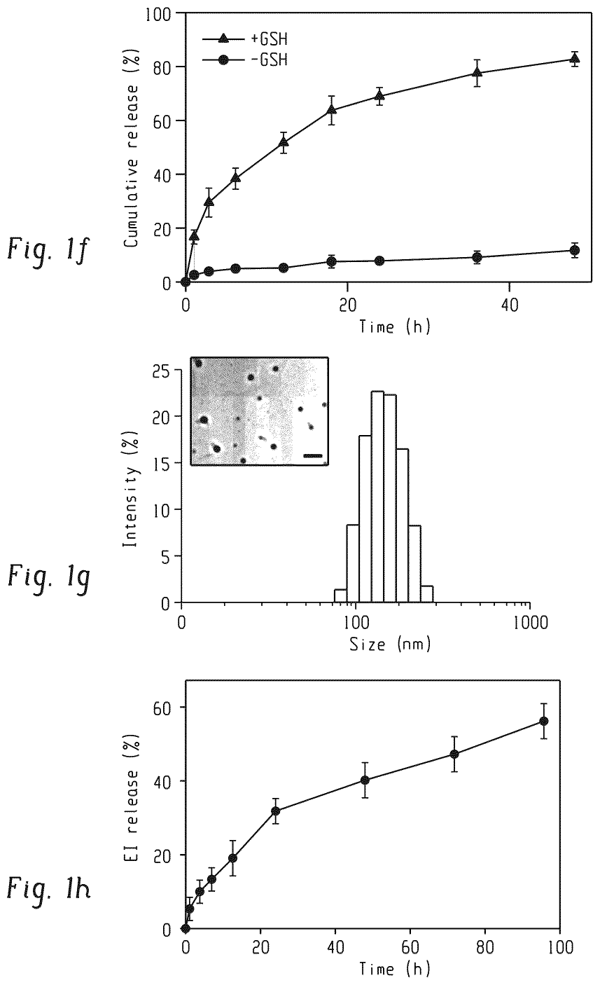

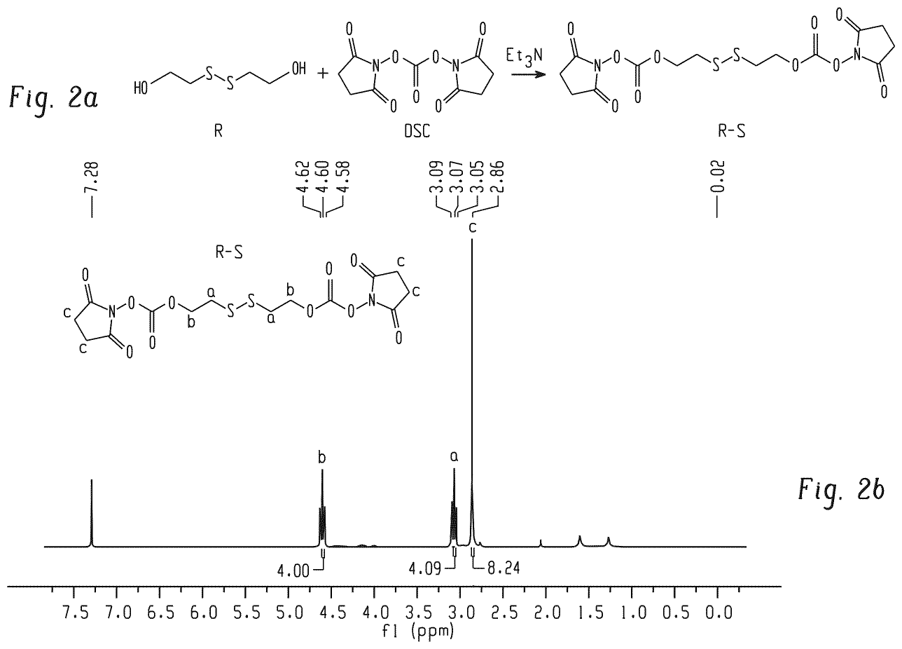

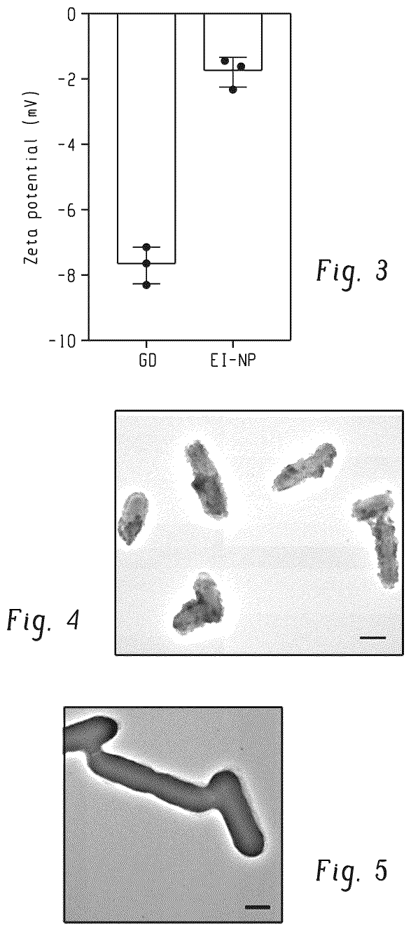

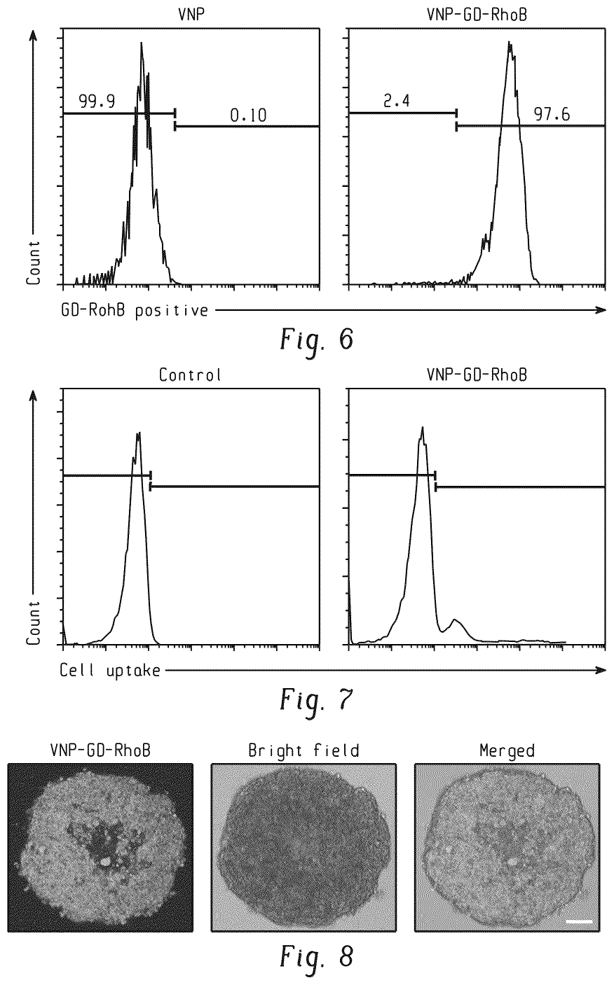

Cells tend to maintain redox homeostasis; the oxidative stress in tumor tissue usually causes higher glutathione (GSH) expression in tumor cells than that in normal cells. Therefore, to achieve selective protein release inside tumor cells, a GSH responsive linker (FIG. 2) was synthesized to crosslink GSDMD proteins to form protein cages (designated GD) that can be easily modified on the surface of VNP. Furthermore, as shown in FIG. 1c and FIG. 3, GSDMD proteins were assembled into protein cages with the particle size of about 130 nm and the zeta potential of about −7.7 mV, through crosslinking with GSH responsive linkers. Typically, negatively charged free GSDMD proteins or protein cages are hard to transport into tumor cells to initiate pyroptosis. In this regard, VNP20009 (VNP) was selected as a carrier to shuttle GSDMD to the cytoplasm of tumor cells. VNP is a genetically modified strain of attenuated Salmonella typhimurium with a good biosafety profile as is known in the art. As an intracellular bacterium, flagellum enabled strong mobility and natural tropism towards tumor hypoxic areas make VNP an excellent carrier for tumor-selective and intracellular delivery of protein cargoes. Moreover, the abundant flagella of VNP could activate the caspase 1 to transform the delivered GSDMD proteins into active form for triggering tumor cell pyroptosis. After removing extra linkers by ultrafiltration, the GSDMD protein cages were conjugated on the surface of VNP by a facile amide reaction. The obvious particle protrusions were visualized on the surface of the modified bacteria under transmission electron microscope (TEM) imaging (FIG. 1d, FIG. 4) compared to the unmodified bacteria (FIG. 5). Furthermore, the successful conjugation was verified by the colocalization of the Hoechst-labeled bacteria and Rhodamine B-labeled protein cages (FIG. 1e). According to the flow cytometry assay, over 97% of the bacteria were conjugated with protein cages (FIG. 6). Subsequently, the protein release behavior of the protein cage conjugated bacteria delivery system was evaluated; the presence of 10 mM GSH (typically representing the intracellular GSH level in tumor cells) significantly increased the protein release rate, with over 60% of protein released within 20 hours compared to that without GSH (FIG. 1f).

Under normal circumstances, the Ca²⁺ concentration outside the cell is much higher than the intracellular Ca²⁺ concentration, and the maintenance of this concentration gradient is mainly governed by the calcium ion channel on the cell surface. However, after pyroptosis-induced pore formation and cell membrane damage, the calcium influx would trigger the ESCRT-mediated cell membrane repair. To inhibit the calcium influx triggered ESCRT cell membrane repair for improving VNP-GD-induced tumor cell pyroptosis, a biocompatible dextran nanoparticle (EI-NP) loaded with the ESCRT inhibitor (a potent calcium ion antagonist, BAPTA-AM) was formulated, showing the monodispersed spherical structure under TEM observation with a particle size of approximately 150 nm and the zeta potential of −1.8 mV (FIG. 1g, FIG. 3). Moreover, over 58% of the ESCRT inhibitor was released from the dextran nanoparticle within 96 h under pH 6.5 condition that mimics the pH level in the tumor microenvironment (FIG. 1h).

### Example 2: Characterization of VNP-GD-Induced Tumor Cell Pyroptosis

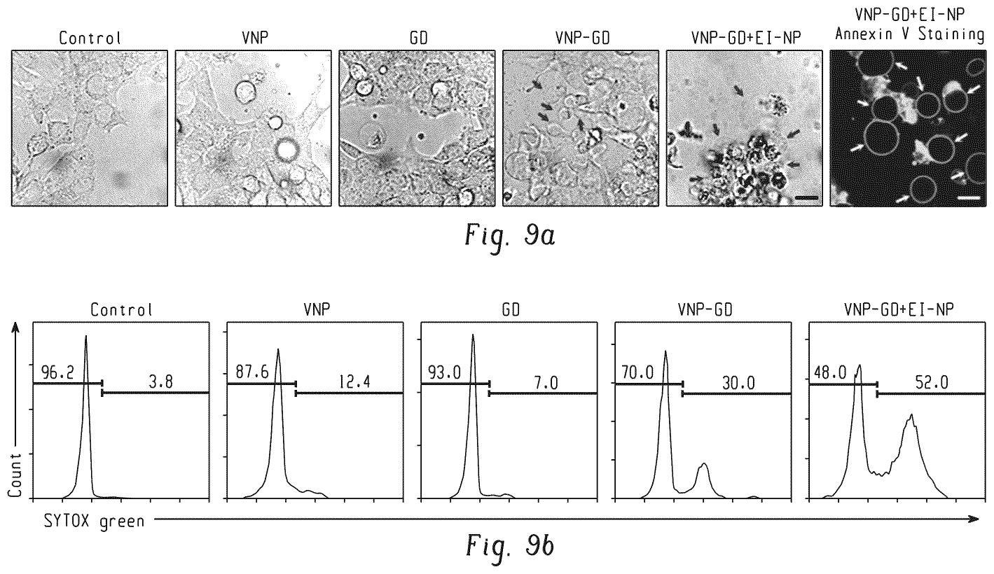

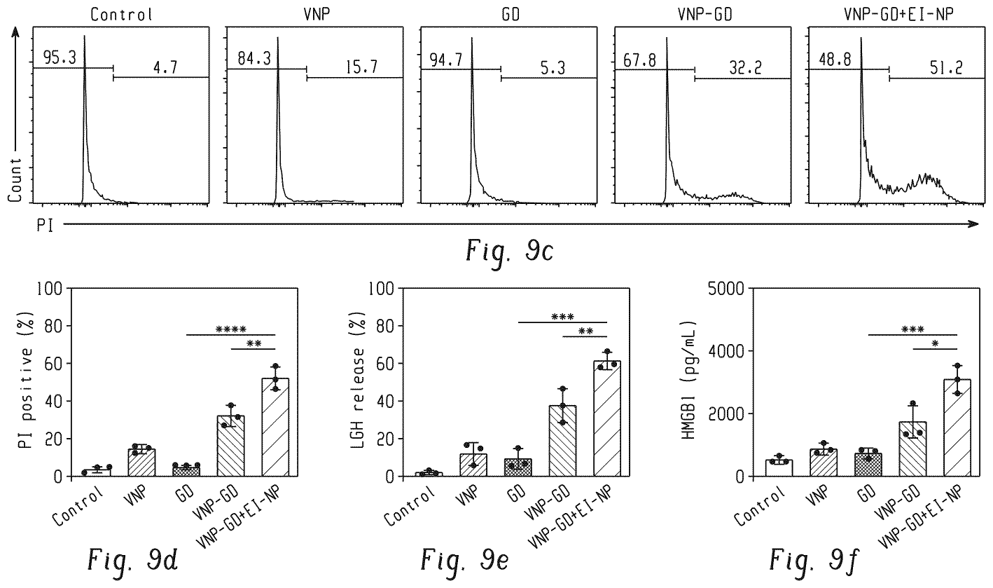

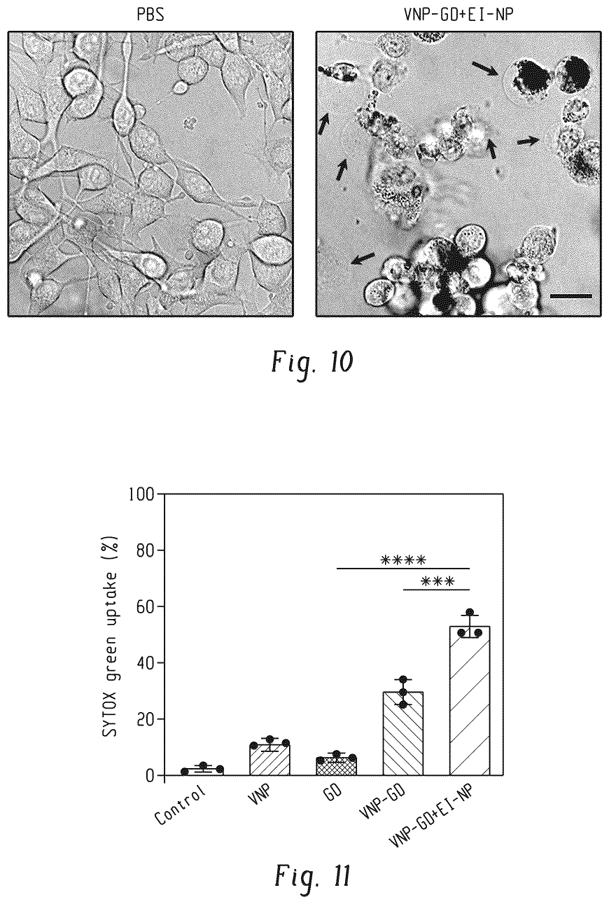

The good biosafety and tumor-targeting tendency of VNP makes it an excellent vector to deliver therapeutic proteins directly to tumors. As an intracellular bacteria strain, VNP efficiently delivered GD protein cages into 4T1 cells (FIG. 7). Moreover, because of its hypoxia tropism and abundant flagella-enabled strong motility, VNP-GD showed excellent tumor penetration ability in 4T1 3D tumor cell sphere (FIG. 8). After the verification of the successful intracellular GSDMD protein delivery, next the efficacy of VNP-GD in triggering tumor cell pyroptosis was evaluated in both 4T1 and B16F10 tumor cells. Pyroptosis is typically characterized by the expansion of cells forming large ballooning bubbles before the cell membrane ruptures eventually. Thus, the cellular morphology after different treatments was first observed under a confocal microscope, and the transparent large ballooning bubbles forming in 4T1 cells and B16F10 cells were found after the treatments of VNP-GD and VNP-GD+EI-NP (FIG. 9a, FIG. 10). Moreover, blocking ESCRT III-mediated membrane repair by EI-NP substantially increased the number of pyroptotic tumor cells generated by VNP-GD. However, negligible tumor pyroptosis was observed in either VNP group or GD group, suggesting that the initiation of tumor cell pyroptosis requires the presence of both VNP and GD.

SYTOX® green is a nucleic acid dye that can be applied to characterize the cell membrane integrity since it can only pass through damaged cytoplasmic membranes to bind the nucleic acid in which its fluorescence intensity will be significantly enhanced after binding. Therefore, the cell uptake of SYTOX® green could serve as an indication of tumor cell pyroptosis after different treatments. It was found that the VNP-GD increased the cell uptake of SYTOX green from 3.8% (control group) to 30%. Notably, VNP-GD+EI-NP treatment significantly enhanced the cell uptake of SYTOX green to 52%, suggesting the strongest pyroptosis-induced cell membrane rupture and pore formation among all treatment groups (FIG. 9b, FIG. 11). Moreover, the propidium iodide (PI) staining was performed to further verify the programmed cell death and cell membrane damage. As shown in FIG. 9c, d, the percentage of PI-positive cells in VNP-GD+EI-NP group was 51.2% which was significantly higher than that in the VNP-GD (32.2%), VNP (15.7%), and GD (5.33%) groups. During cell pyroptosis, the activated N-terminal GSDMD proteins would insert into the cell membrane to form the pore, and eventually the cells rupture to release the inside contents, which could result in elevated lactate dehydrogenase (LDH) released from tumor cells. Therefore, LDH release was detected to evaluate the cytotoxicity of the VNP-GD+EI-NP against tumor cells. It was found that after blocking the ESCRT mediated cell membrane repair, the LDH release in VNP-GD+EI-NP group was 1.60-fold higher than that in VNP-GD group (FIG. 9e). Furthermore, as a typical intracellular tumor antigen, the high mobility group box 1 protein (HMGB1) release was also detected by the enzyme-linked immunosorbent assay (ELISA) kit, and the VNP-GD+EI-NP treatment increased HMGB1 expression from 583 μg/ml (control group) to 3083 μg/ml, which was 1.75-fold higher than that of VNP-GD (FIG. 9f), suggesting the enhanced tumor antigen release after the VNP-GD+EI-NP treatment that was attributed to the induced tumor cell pyroptosis.

### Example 3: Mechanistic Study of Tumor Pyroptosis and ESCRT-Dependent Cell Membrane Repair

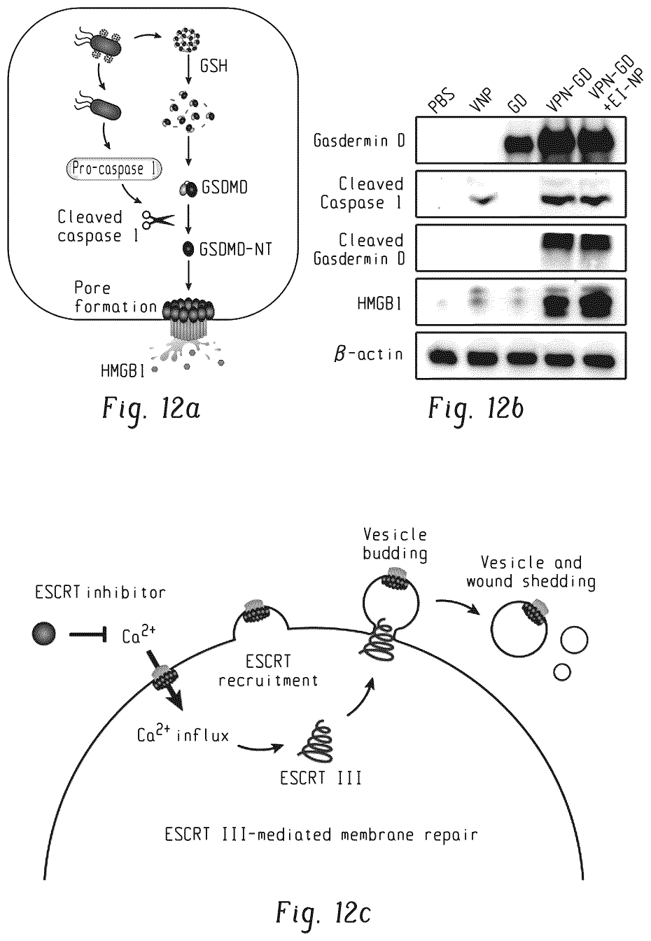

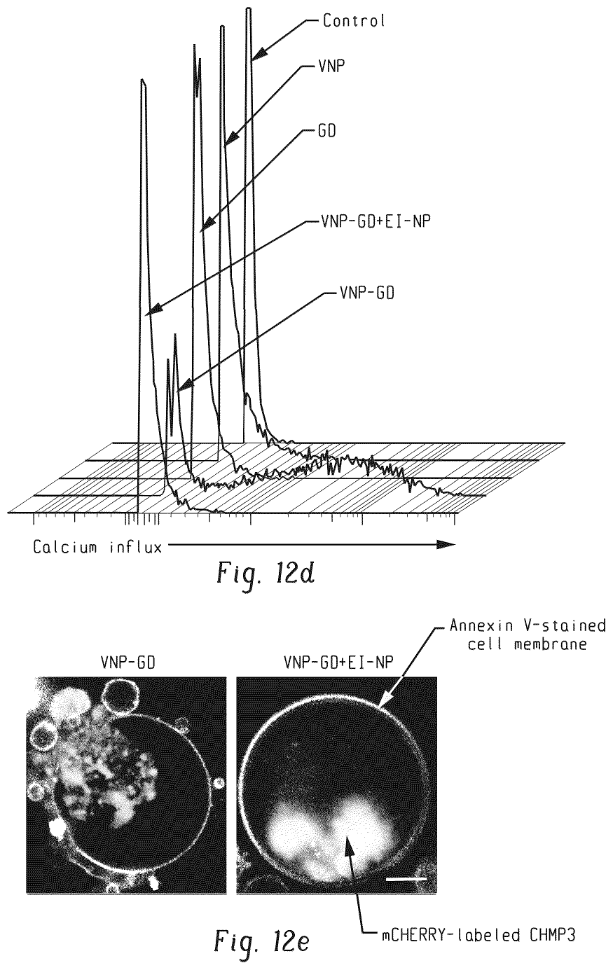

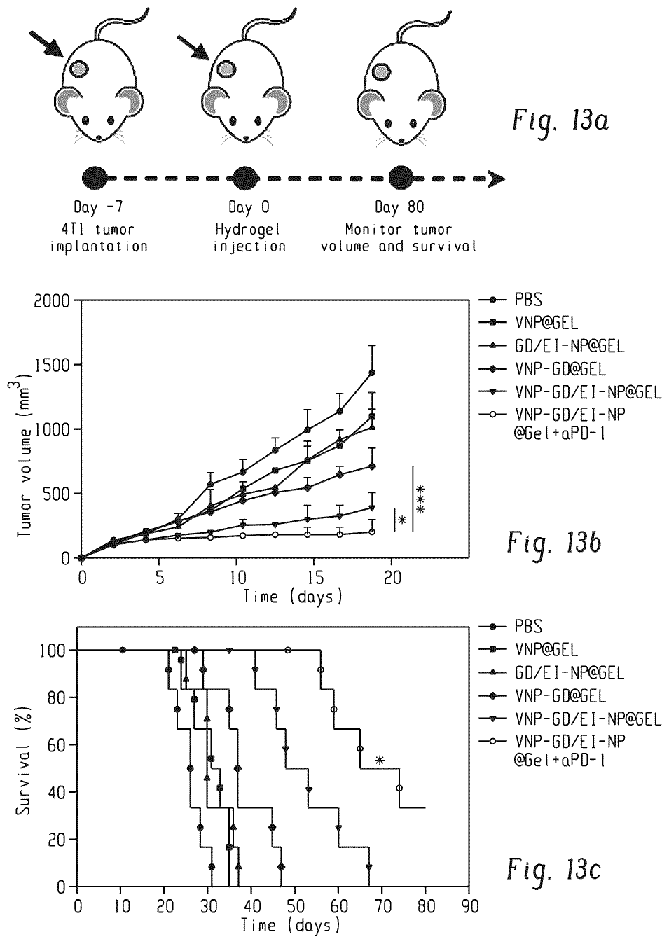

GSDMD protein-induced pyroptosis signaling pathway is mainly illuminated in immune cells and remains elusive in tumor cells. To reveal the underlying mechanism of VNP-GD-triggered tumor cell pyroptosis, the western blot assay was first performed to investigate the key protein expressions in the activated tumor cell pyroptosis signaling pathway. As shown in FIG. 12a, b, 4T1 tumor cells without any treatment showed negligible GSDMD protein expression, while VNP-GD could efficiently deliver GSDMD to the cytoplasm of 4T1 cells. Furthermore, it was found that VNP could activate the caspase 1 to cleaved caspase 1, which is probably attributed to the flagella on the surface of the bacteria. However, without the VNP treatment, there is negligible cleaved caspase 1 expression in PBS and GD groups, demonstrating the crucial role of VNP in activating the caspase 1 signaling pathway. Next, the cleaved GSDMD proteins (N-terminal GSDMD) were observed in both VNP-GD and VNP-GD+EI-NP groups, and there was no significant difference between these two groups, indicating that the enhanced tumor cell pyroptosis by EI-NP treatment was not through increasing the cleavage of GSDMD. Moreover, there was GSDMD expression detected in the GD group, but no cleaved GSDMD was detected, which revealed that GSDMD could not be activated to induce further pyroptosis without VNP-mediated caspase 1 cleavage. Subsequently, after the GSDMD cleavage, the N-terminal GSDMD will bind to phospholipid proteins on the cell membrane to form holes to induce pyroptosis, accompanied with the release of intracellular inflammatory antigens. Notably, compared with the VNP-GD group, a substantially increased HMGB1 expression was observed in the VNP-GD+EI-NP group, suggesting stronger cell membrane rupture generated by VNP-GD+EI-NP treatment. Collectively, it was demonstrated here that VNP-GD could efficiently trigger tumor cell pyroptosis by the combination of enhancing intracellular delivery of GSDMD via VNP and further activation of N-terminal GSDMD mediated by VNP-induced caspase 1 cleavage. Moreover, the addition of EI-NP enhanced the pyroptosis as evidenced by the increased generation of tumor antigen HMGB1 but did not alter the expression of cleaved caspase 1 or cleaved GSDMD.

To investigate the EI-NP-mediated pyroptosis enhancement, the ESCRT III machinery-mediated membrane repair was investigated after VNP-GD+EI-NP treatment. The calcium influx, the initiator of ESCRT III-induced membrane repair, was first evaluated to verify if pyroptosis could trigger calcium influx and if EI-NP could inhibit the calcium influx in the 4T1 tumor cells. As shown in FIG. 12c, d, VNP-GD could trigger the calcium influx as indicated by an obvious increase of the fluorescence intensity of the green fluorescent calcium-binding dye (Fluo-8 AM), which was probably attributed to the pyroptosis induced pore formation or cell membrane damage. However, after treatment with the VNP-GD+EI-NP group, there was no significant increase in the fluorescence intensity compared to the control, VNP and GD groups, suggesting the blocked calcium influx by the EI-NP. Moreover, the role of ESCRT III machinery in tumor cell membrane repair during pyroptosis was investigated with the hypothesis of blocking calcium influx to prevent the recruitment of ESCRT III machinery. Charged multivesicular body protein 3 (CHMP3) is a main functional part of ESCRT III machinery during cell membrane repair, therefore, a mouse full-length-CHMP3-mCherry plasmid was constructed and transfected into 4T1 cells to obtain the CHMP3-mCherry-expressing 4T1 cells. As shown in FIG. 12e, the mCherry-labeled CHMP3 was located on the cell membrane and formed microvesicles to fix the pores and help the cell membrane repair during VNP-GD-triggered tumor pyroptosis. Notably, after the treatment of VNP-GD+EI-NP, the mCherry-labeled CHMP3 proteins were mainly distributed in the cytoplasm and not involved in the cell membrane repair with negligible microvesicles formed during pyroptosis, which was attributed to the prevention of the recruitment of ESCRT III machinery by blocking calcium influx. It was demonstrated that ESCRT III-mediated membrane repair could increase the resistance of 4T1 tumor cells to pyroptosis through sewing the pore, budding and forming microvesicle, thus blocking ESCRT III-mediated membrane repair via preventing calcium influx could enhance tumor pyroptosis.

### Example 4: Anti-Tumor Efficacy of VNP-GD+EI-NP in 4T1 Metastatic Triple-Negative Breast Cancer and B16F10 Melanoma

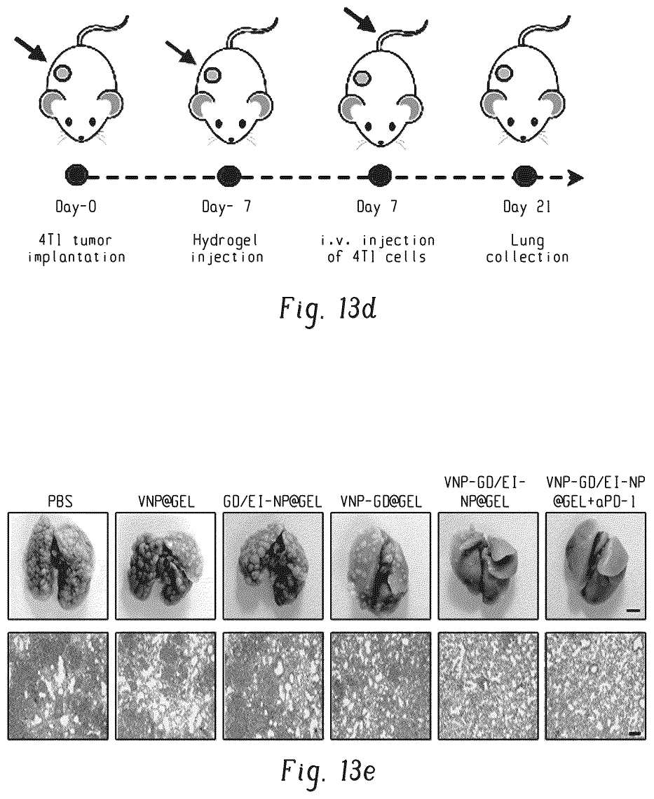

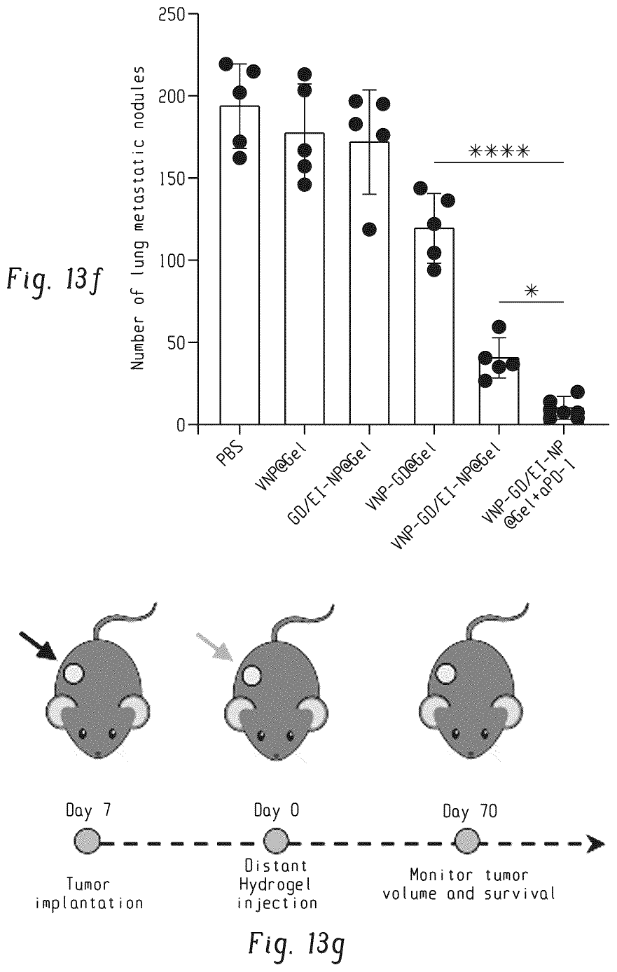

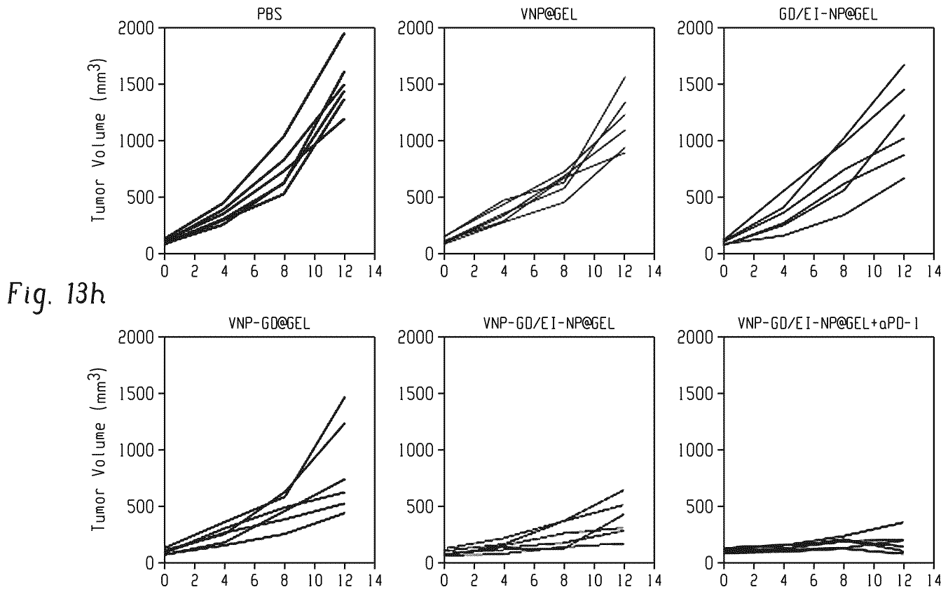

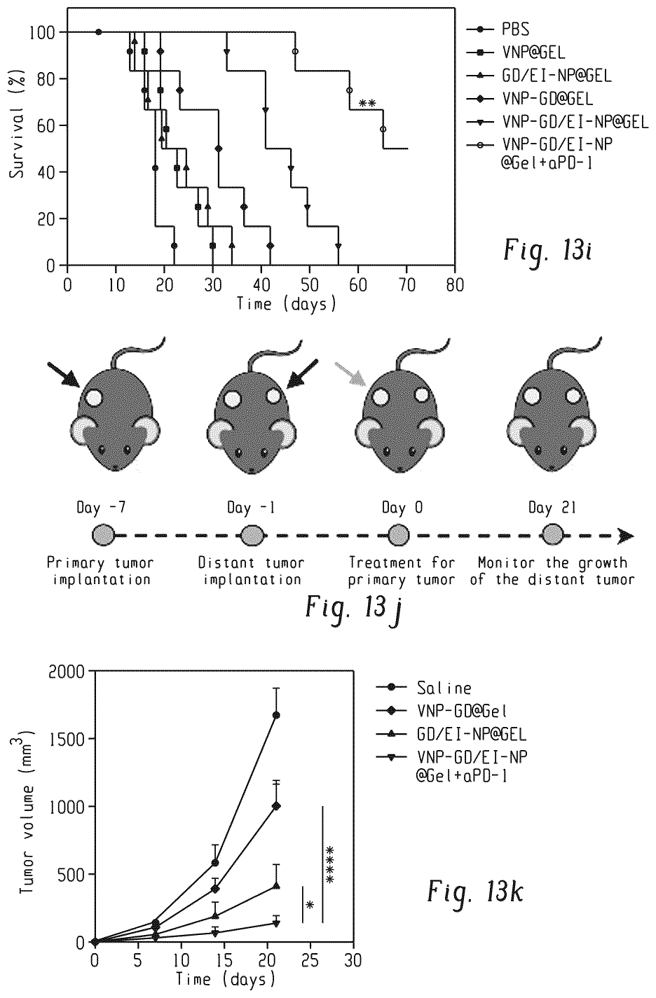

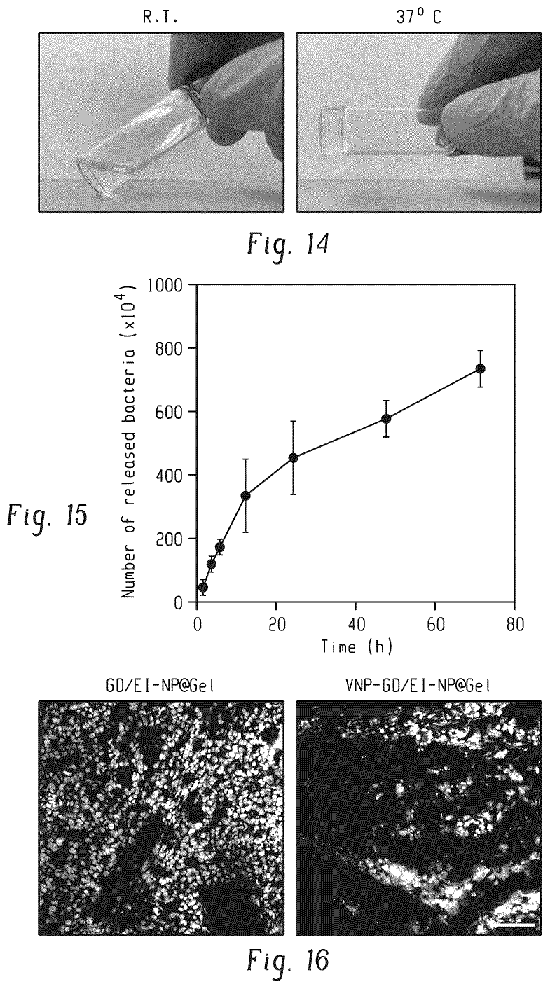

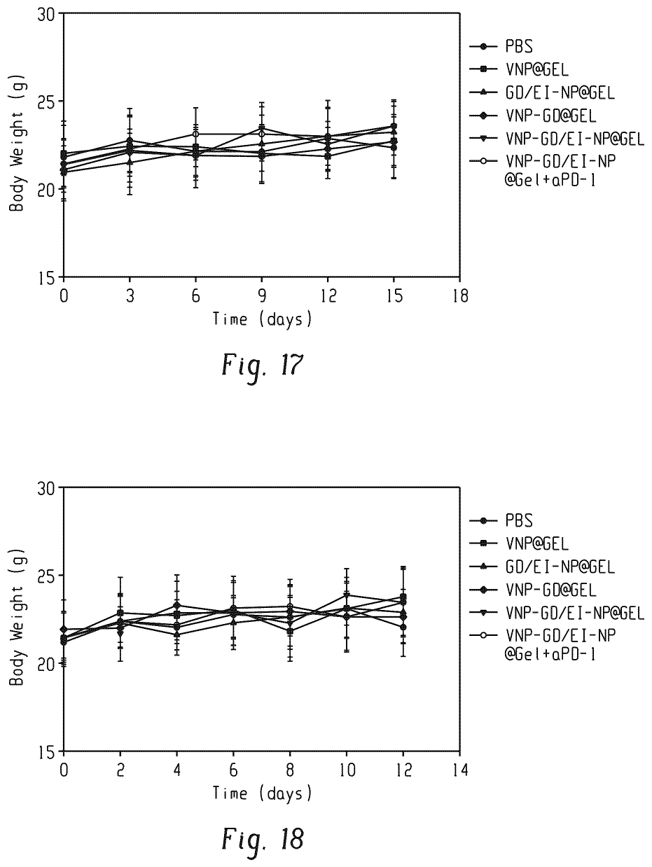

The in vivo pyroptosis-triggered tumor immunotherapy efficacy of VNP-GD and EI-NP was first evaluated on a 4T1 breast cancer model (FIG. 13a). To enable the in vivo delivery of VNP-GD and EI-NP, an injectable thermoresponsive Pluronic® F-127-based hydrogel was developed for peritumoral administration. VNP-GD and EI-NP were co-loaded into the Pluronic F127 solution at room temperature, which formed in a hydrogel for about 1 minute at 37° C. through a sol-gel transition (FIG. 14). Additionally, the release of the bacteria from the hydrogel was evaluated, and more than 65% of the VNP-GD was released within 72 h (FIG. 15). To further characterize the tumoral distribution of VNP-GD after peritumoral administration, GSDMD proteins were labeled with Rhodamine B for tumor tissue imaging. As shown in FIG. 16, in the VNP-GD/EI-NP@Gel group, there were more Rhodamine B-labeled GD proteins distributed in the tumor than that in the GD/EI-NP@Gel group which could be attributed to the strong motility and hypoxia tropism of VNP improving the tumoral delivery and penetration of GD proteins.

As shown in FIG. 13b, VNP-GD/EI-NP@Gel (hydrogel co-loaded with VNP-GD and EI-NP) exhibited superior anti-tumor efficacy than VNP-GD@Gel and displayed the best tumor growth inhibition when combined with aPD-1 antibodies (VNP-GD/EI-NP@Gel+aPD-1 group). The average tumor volume in the VNP-GD/EI-NP@Gel+aPD-1 group on day 18 was 2- and 3.7-fold smaller than that in VNP-GD/EI-NP@Gel and VNP-GD@Gel groups, respectively. Additionally, the VNP-GD/EI-NP@Gel+aPD-1 prolonged the survival time of the 4T1 tumor-bearing mice with a median survival of 69.5 days, which was significantly longer than that of VNP-GD/EI-NP@Gel (50.5 days), indicating that the immune checkpoint blockade could synergize with the pyroptosis-mediated immune response to improve anti-tumor immunotherapy efficacy (FIG. 13c). Notably, compared with VNP-GD@Gel group (median survival, 37 days), VNP-GD/EI-NP@Gel treatment achieved better anti-tumor efficacy, demonstrating the importance of blocking ESCRT III-mediated cell membrane repair for enhancing tumor cell pyroptosis. Considering that lung metastasis is closely associated with breast cancer patients in the clinic and causing high mortality, we further applied VNP-GD/EI-NP@Gel+aPD-1 treatment strategy on a 4T1 breast cancer lung metastasis model. In an established 4T1 breast cancer lung metastasis model (FIG. 13d), it was demonstrated that VNP-GD/EI-NP@Gel+aPD-1 potently inhibited lung metastasis and decreased the number of lung metastatic nodules compared to both VNP-GD@Gel and VNP-GD/EI-NP@Gel groups, as evidenced by the lung images and H&E stainings (FIG. 13e, 13f). Notably, the number of surface lung metastasis nodules in the VNP-GD@Gel group was 2.9-fold higher than that in the VNP-GD/EI-NP@Gel group, suggesting that enhancing pyroptosis by blocking ESCRT-mediated cell membrane repair holds great potential to prevent lung metastasis.

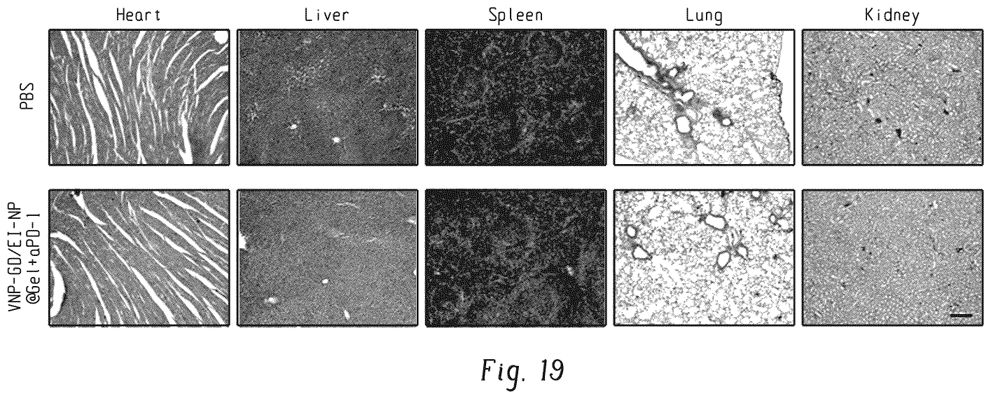

Moreover, to validate the broad applicability of the VNP-GD/EI-NP@Gel+aPD-1 treatment strategy, a B16F10 melanoma tumor model was established and treated with various formulations (FIG. 13g). As shown in FIG. 13h, the B16F10 tumors in the PBS control group displayed a sharp increase in the tumor volumes with all over 1000 mm³ on day 12, while both the VNP@Gel and GD/EI-NP@Gel showed the slight inhibition of the tumor growth. Notably, the tumor growth in the VNP-GD/EI-NP@Gel group was much slower than that in the VNP-GD@Gel group, which highlighted the importance of EI-NP in improving tumor pyroptosis-induced anti-tumor immune response. The best treatment efficacy of VNP-GD/EI-NP@Gel+aPD1 was further substantiated, as evidenced by the smallest tumor volumes among all the treatment groups (FIG. 13h). Moreover, VNP-GD/EI-NP@Gel+aPD1 prolonged the median survival of the tumor-bearing mice from 18 days (PBS) to 67.5 days, while VNP@Gel and GD/EI-NP@Gel only prolonged the median survival to 22 days and 22.5 days respectively. Notably, the more prolonged median survival of VNP-GD/EI-NP@Gel (43.5 days) compared with VNP-GD@Gel (31 days) further demonstrated that blocking ESCRT mediated cell membrane repair could enhance tumor pyroptosis-mediated anti-tumor efficacy (FIG. 13i). Furthermore, to investigate if this local treatment could induce a systemic immune response, a distant tumor model was established as illustrated in FIG. 13j. The VNP-GD/EI-NP@Gel treatment showed a better tumor inhibition than the VNP-GD@Gel treatment group indicating a stronger systemic immune activation induced by the pyroptosis-mediated tumor antigen release that resulted from the combination of GSDMD-induced and ESCRT III inhibition-boosted tumor cell pyroptosis. In addition, the distant tumor volumes in the VNP-GD/EI-NP@Gel+aPD-1 group were significantly smaller than that in the VNP-GD/EI-NP@Gel group, demonstrating the synergistic efficacy when combined with aPD-1 antibodies to block PD1/PDL1 pathway to re-activate T cells for anti-tumor immunity (FIG. 13k). Moreover, both in the 4T1 tumor model and B16F10 tumor model, the body weight of the mice showed no significant decrease after the treatment of the hydrogel-based delivery systems (FIG. 17, FIG. 18). In addition, H&E assay of the major organs in the C57BL6 mice further demonstrated a good biosafety profile of the hydrogel-based delivery system (FIG. 19).

### Example 5: Preventing ESCRT-Dependent Cell Membrane Repair Enhanced Pyroptosis and Augmented Anti-Tumor Immune Response

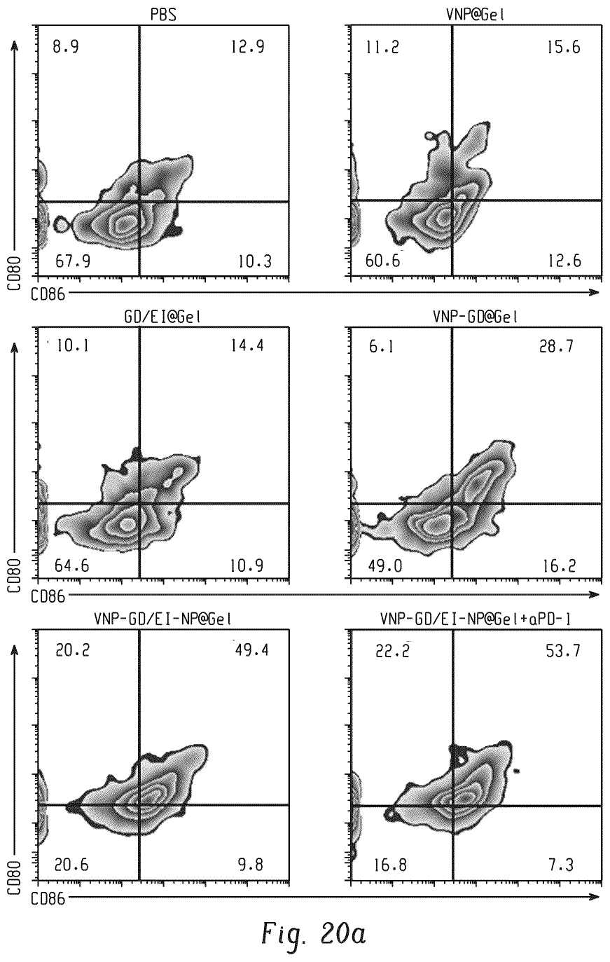

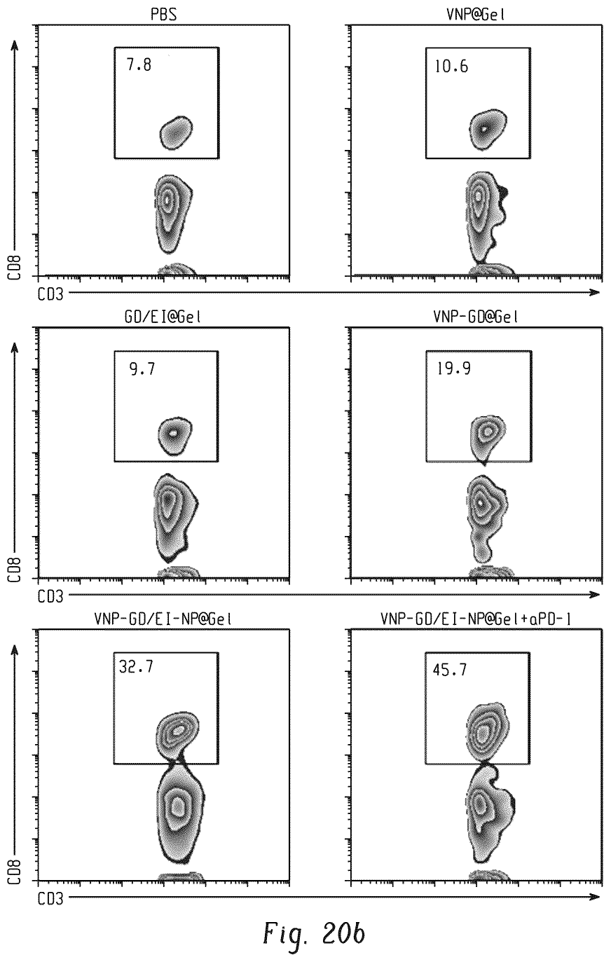

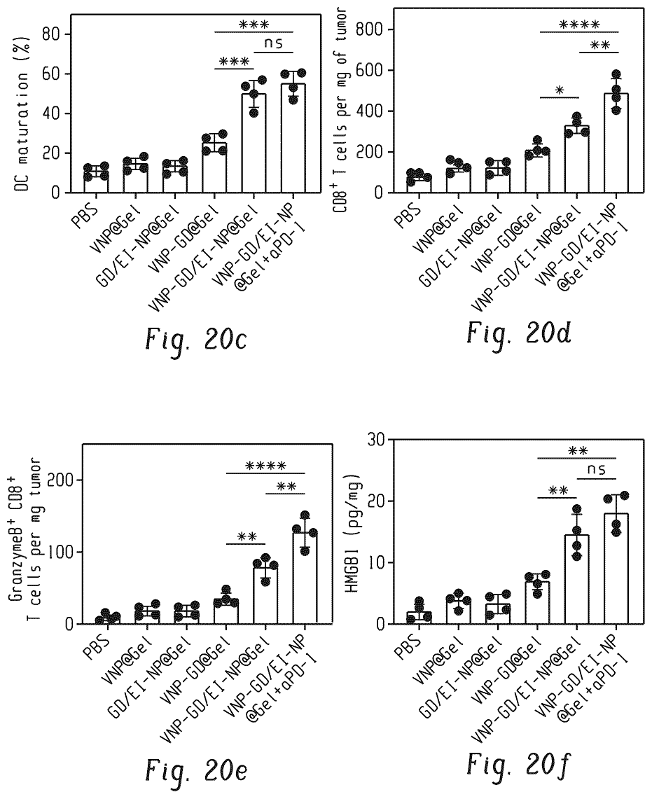

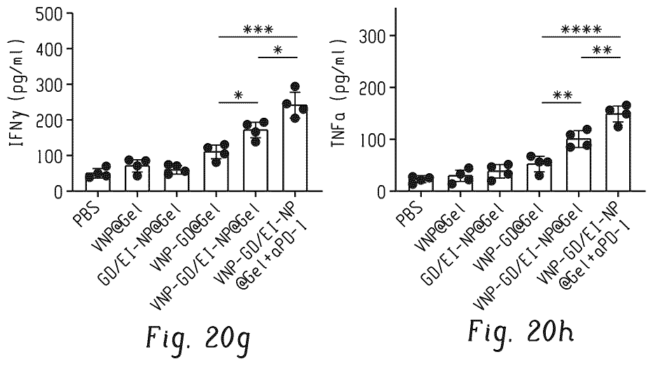

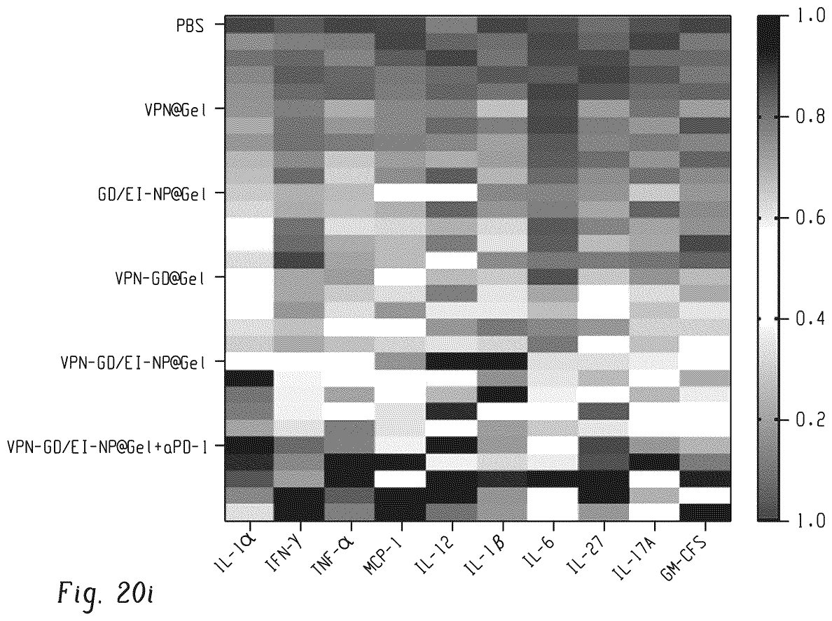

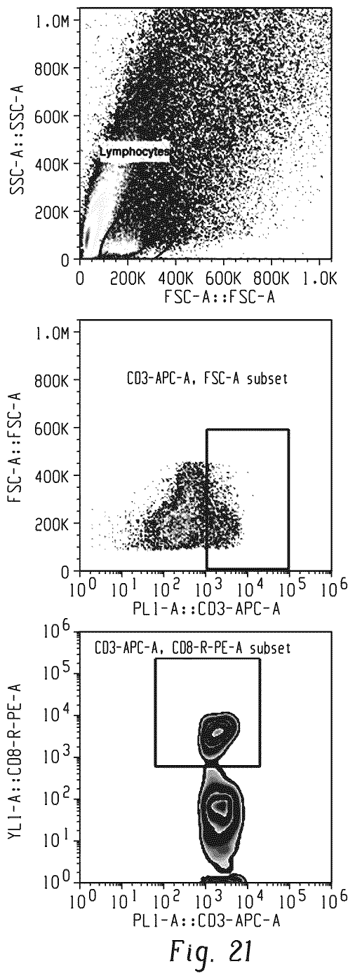

Pyroptosis is a form of programmed cell death that produces a large number of inflammatory factors, releases tumor antigens, and initiates antigen-presenting cells (APC)-mediated adaptive immune responses, which could be leveraged to reawaken the immune system and overcome the tumor immunosuppressive microenvironment. Therefore, the dendritic cell (DC) maturation was first investigated after different treatments, and it was found that VNP-GD@Gel increased the CD80⁺CD86⁺ proportion of DCs from 12.9% (PBS group) to 28.7%, while VNP-GD/EI-NP@Gel increased the DC maturation to 49.4%, significantly higher than that of VNP-GD@Gel group (FIG. 20a, c). The increased DC maturation in VNP-GD/EI-NP@Gel was probably attributed to the enhanced tumor antigen release after tumor cell pyroptosis mediated by the combination of tumor-selective intracellular delivery of GSDMD by VNP and membrane repair inhibition by EI-NP. Next, the CD8⁺ T cell infiltration at the tumor site was evaluated, and it was found that the VNP-GD/EI-NP@Gel+aPD-1 treatment increased the CD8⁺ T cell infiltration from 7.8% (PBS group) to 45.7%, significantly higher than that of the VNP-GD@Gel (19.9%) and VNP-GD/EI-NP@Gel (32.7%) (FIG. 20b, FIG. 21). More specifically, the infiltrated CD8⁺ T cell number increased from approximately 76 per mg tumor (PBS group) to approximately 493 per mg tumor after the VNP-GD/EI-NP@Gel+aPD-1 treatment (FIG. 20d). Moreover, VNP-GD/EI-NP@Gel+aPD-1 also potently increased the number of activated Granzyme B+CD8⁺ T cells in the tumor, which was significantly higher than that in the VNP-GD@Gel and VNP-GD/EI-NP@Gel groups (FIG. 20e).

Furthermore, according to the ELISA assay of HMGB1 expression in FIG. 20f, compared with the PBS group, VNP@Gel and GD/EI-NP@Gel slightly increased the HMGB1 expressions in the tumor tissues, while the VNP-GD@Gel displayed a 3-fold increase of HMGB1 expression. Notably, after blocking the ESCRT-based cell membrane repair through EI-NP, VNP-GD/EI-NP@Gel and VNP-GD/EI-NP@Gel+aPD-1 treatments showed 7.1- and 8.1-fold increases in HMGB1 expression, respectively, when compared to PBS group, which was also significantly higher than that of VNP-GD@Gel group. Moreover, as shown in FIG. 20g, VNP-GD@Gel treatment resulted in a 2.2-fold increase of IFNγ expression in the tumor tissue, while the VNP-GD/EI-NP@Gel treatment showed a 3.5-fold increase of IFNγ expression when compared to that in PBS group. Moreover, after combination with immune checkpoint blockade therapy, VNP-GD/EI-NP@Gel+aPD-1 treatment showed the highest IFNγ expression level among all treatment groups. Similarly, VNP-GD/EI-NP@Gel+aPD-1 also increased the TNFα expression, which was 1.5-fold higher than that of VNP-GD/EI-NP@Gel treatment and 2.7-fold higher than that of VNP-GD@Gel treatment (FIG. 20h). Furthermore, as shown in FIG. 20i, the potently elevated inflammatory cytokines and chemokines expression detected by LEGENDplex™ multi-Analyte Flow assay kit after the VNP-GD/EI-NP@Gel+aPD-1 treatment further substantiated the strongest immune activation among all treatment groups.

### Example 6: Lyophilized Hydrogel-Based Cell Patch for the Treatment of Inoperable Ovarian Cancer

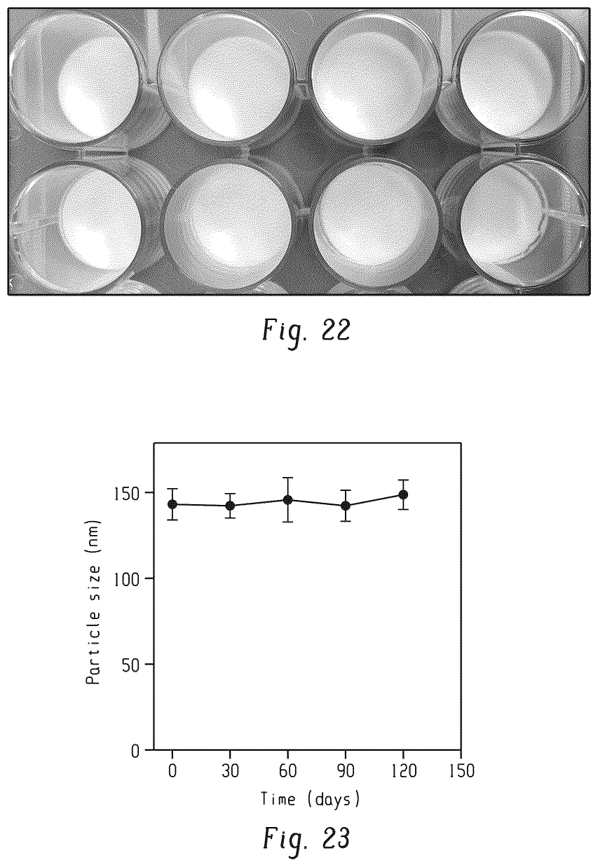

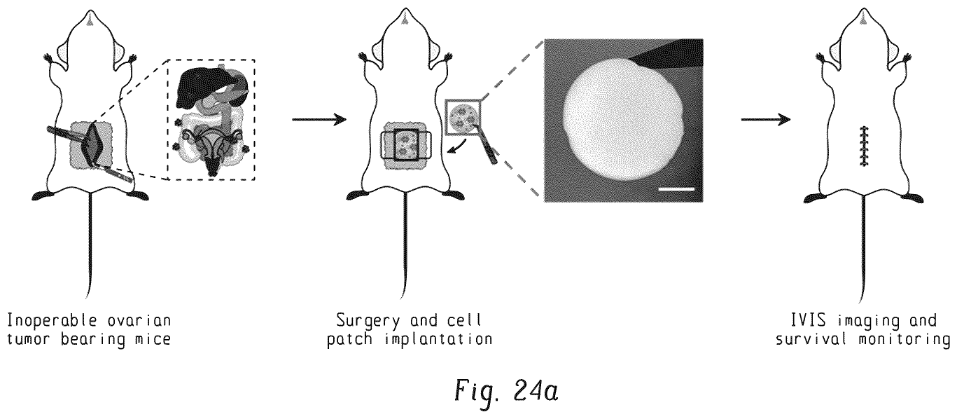

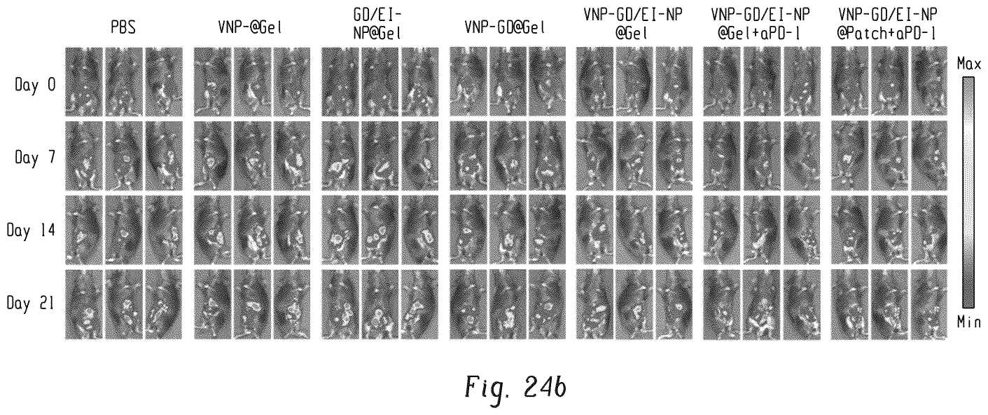

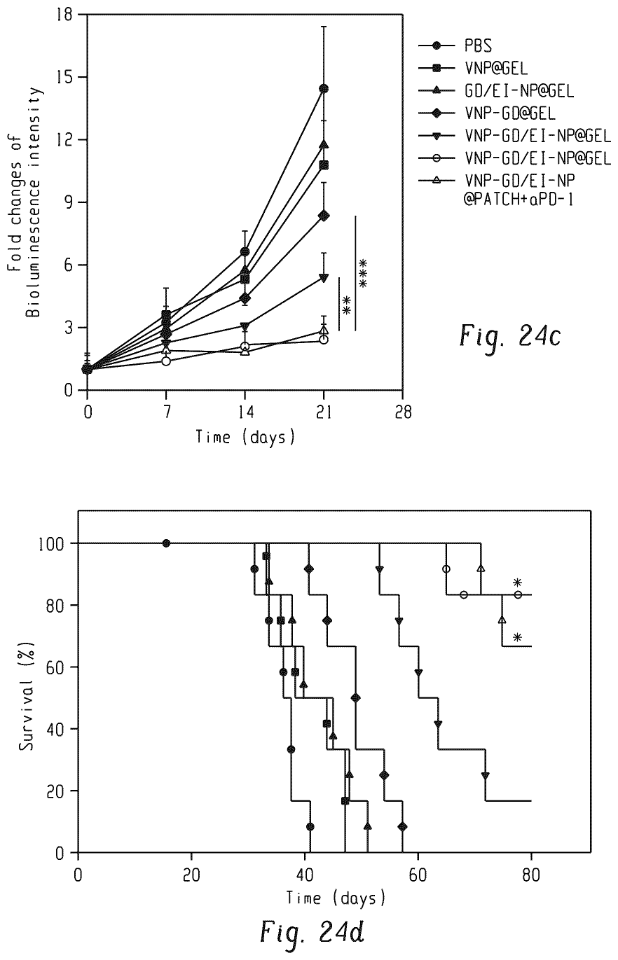

Ovarian cancer is a highly malignant tumor, and because of the lack of typical clinical symptoms in the early stage, many patients are associated with a large number of organ metastases in the abdominal cavity, including metastases in the liver, spleen, and kidney when diagnosed, which makes surgical treatment impossible. Therefore, in view of this clinical challenge, for inoperable tumor treatment, such as advanced metastatic ovarian cancer, a pyroptosis-enhancing cell patch was prepared with a lyophilized hyaluronan hydrogel loaded with EI-NP (FIG. 22). The lyophilized EI-NP showed great stability with negligible particle size change after 120 days (FIG. 23), and the lyophilized hydrogel patch preloaded with EI-NP could serve as an off-the-shelf product for long-term storage. This patch could be easily implanted into the abdominal cavity after the addition of freshly prepared VNP-GD to treat metastatic ovarian cancer (FIG. 24a). As shown in FIG. 24b, c, the bioluminescence intensity from luciferase-expressing ID8 ovarian cancer cells in the VNP-GD/EI-NP@Gel group was significantly weaker than that in the VNP-GD@Gel group. Moreover, VNP-GD/EI-NP@Gel+aPD-1 and VNP-GD/EI-NP@Patch+aPD-1 showed similar anti-tumor efficacy, and both are much better than VNP-GD/EI-NP@Gel and VNP-GD@Gel treatments, suggesting the comparable and potent anti-tumor treatment efficacy of lyophilized cell patches. More importantly, VNP-GD/EI-NP@Gel prolonged the median survival of the inoperable ovarian tumor-bearing mice from 37.5 days (PBS group) to 60.5 days, which was significantly longer than that of VNP-GD@Gel group (49 days), demonstrating the importance of ESCRT inhibition in the pyroptosis-mediated cancer immunotherapy (FIG. 24d). VNP-GD/EI-NP@Gel+aPD-1 and VNP-GD/EI-NP@Patch+aPD-1 showed similar prolongation of the survival time, and over 65% of mice were still alive after 80 days indicating that the combination of immune checkpoint blockade with tumor pyroptosis could synergistically enhance anti-tumor immunity to improve treatment outcomes.

### Discussion

Pyroptosis, an inflammatory cell necrosis that has evolved from bacterial infection-triggered caspase-1 dependent cell death to gasdermin-dependent programmed cell death, has attracted extensive attention from basic research to disease treatment. For instance, inhibiting immune cell pyroptosis can be utilized to treat inflammatory diseases such as cardiovascular disease and sepsis, while promoting cell pyroptosis can be used for anti-tumor immunotherapy. However, for tumor cell pyroptosis, overexpression of DNA methyltransferase restricted the expression of gasdermins, especially GSDME (DFNA5), prevents the application of leveraging gasdermin-dependent tumor cell pyroptosis for anti-tumor treatment. To overcome this, the integration of DNA methyltransferase inhibitors that can increase the GSDME expression with chemical drugs that can activate the caspase-3 pathway to cleave GSDME for perforating membrane, has been suggested for triggering GSDME-mediated tumor pyroptosis. Despite GSDME protein, other gasdermin family proteins, including GSDMA, GSDMB, GSDMC, and GSDMD, have been found to trigger cell pyroptosis through an activated pore-forming domain unleashed by the distinct enzyme initiator. Even though there are extensive mechanistic investigations, few have entered in vivo studies, particularly for anti-tumor applications. The reasons accounting for the lack of in vivo anti-tumor evaluations include the relatively restricted Gasdermin protein expression in tumor cells, the complication of initiating gasdermin-mediated tumor cell pyroptosis requiring activation of multiple signaling pathways to unleash the pore-forming domain of Gasdermin protein, and the presence of ESCRT III machinery-dependent membrane repair, which work synergistically inhibit the in vivo applications of gasdermin-dependent pyroptosis.

Described herein is a VNP bacteria-based GSDMD protein delivery system for in vivo anti-tumor treatment, in which VNP could efficiently shuttle GSDMD to the intracellular compartment of tumor cells where flagella on VNP activated capapase-1 to further cleave delivered GSDMD to N-terminal domain for effective pore-forming mediated pyroptosis. To overcome ESCRT III machinery-induced cell membrane repair that can compromise the efficacy of tumor cell pyroptosis, a biodegradable nanoparticle was prepared to load Ca²⁺ chelator that can inhibit calcium influx to prevent the recruitment of ESCRT III machinery. Through the combination of VNP-mediated delivery and activation of GSDMD and inhibition of ESCRT III-dependent membrane repair, in vivo anti-tumor treatment efficacy has been demonstrated on multiple tumor models.

Free GSDMD is difficult to diffuse into tumor cells due to their relatively large molecular weight and negative surface charge, and the full length of GSDMD cannot trigger the cell pyroptosis due to the concealment of their active pore-forming domain. To address this challenge, leveraging the superior tumor targeting ability of bacteria delivery systems, we constructed a GSDMD protein cage (GD) with a GSH-responsive linker and further decorated GD on the surface of attenuated Salmonella typhimurium (VNP) to form VNP-GD. As described herein, GSDMD proteins could be efficiently transported to the cytoplasm of tumor cells, in which the VNP could trigger the activation of cleaved caspase 1 to transform intracellularly delivered GSDMD into pore-forming domain N-terminal GSDMD for binding to the plasma membrane and subsequently triggering pyroptosis in tumor cells. Furthermore, ESCRT III mediated membrane repair induced by the calcium influx during pyroptosis could help the tumor cells survive and increase the resistance of tumor cells to pyroptosis. Specifically, after the gasdermin-NT induced pore formation, calcium influx through the pore would trigger ESCRT-mediated membrane repair to subsequently form macrovesicle and stop the continuous expansion of cells and the release of inside antigens, diminishing the treatment efficacy of tumor pyroptosis. To address this issue, described herein is a biocompatible nanoparticle (EI-NP) to bioresponsively release Ca²⁺ chelator to inhibit calcium influx and subsequently prevent ESCRT-dependent membrane repair. In vitro the combination of VNP-GD and EI-NP worked synergistically to initiate and further strengthen the tumor cell pyroptosis, as evidenced by large ballooning bubbles formed and increased release of intracellular contents in both 4T1 and B16F10 cells. To enable in vivo anti-tumor applications, two distinct formulations (an injectable hydrogel and a lyophilized cell patch) were developed for treating multiple tumor settings. In the 4T1 and B16F10 tumor model, the VNP-GD/EI-NP@Gel elicited a strong local and systemic anti-tumor immune response that inhibits the primary and distant tumor growth and prevents the tumor metastasis. Further synergistic treatment efficacy was demonstrated when combining VNP-GD/EI-NP@Gel with systemic injection of immune checkpoint inhibitors, which was probably due to the enhanced tumor antigen release triggered by tumor pyroptosis leading to more T cell infiltration and activation that can be further strengthened through blocking PD1/PDL1 pathway. To treat the clinically inoperable tumor such as late-stage ovarian cancer, a VNP-GD/EI-NP@Gel cell patch was designed and implanted into the abdominal cavity of mice, which significantly inhibited the tumor growth and spread and prolonged the survival time of the tumor-bearing mice.

In summary, described herein is a hydrogel-based delivery system serving as a local reservoir to sustainedly release VNP-GD and EI-NP for enhanced programmed tumor cell death by integrating VNP-activated GSDMD-dependent tumor pyroptosis and inhibition of ESCRT III-mediated plasma membrane repair.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A composition, comprising:
a hydrogel, the hydrogel loaded with
a) bacterial particles comprising a gasdermin D (GSDMD) protein cage conjugated to a surface thereof, and
b) nanoparticles loaded with an ESCRT inhibitor.

2. The composition of claim 1, wherein the GSDMD comprises SEQ ID NO: 1 or SEQ ID NO: 2.

3. The composition of claim 1, wherein the GSDMD protein cage is prepared by crosslinking the GSDMD with a bifunctional linker such as a glutathione (GSH)-responsive linker, a reactive oxygen species (ROS)-responsive linker, a pH-responsive linker, or an enzyme-responsive linker.

4. The composition of claim 1, wherein the bacterial particles comprise attenuated Salmonella, Clostridium, or Bifidobacterium particles, specifically attenuated Salmonella typhimurium particles.

5. The composition of claim 1, wherein the ESCRT inhibitor comprises BAPTA-AM (1,2-Bis(2-aminophenoxy)ethane-N,N,N,N-tetraacetic acid tetrakis(acetoxymethyl ester)), FGI-104 (4-[(7-chloroquinolin-4-yl)amino]-2-(diethylaminomethyl)-6-[4-(hydroxymethyl)-3-methoxyphenyl]phenol), or an RNA interference-mediated inhibitor.

6. The composition of claim 1, wherein the nanoparticles comprise biodegradable nanoparticles such as poly-lactic acid (PLA); poly-D-L-glycolide (PLG); poly-D-L-lactide-co-glycolide (PLGA), poly-alkyl-cyanoacrylate (PCA), poly-ε-caprolactone, gelatin, alginate, chitosan, agarose, polysaccharides, and proteins.

7. The composition of claim 6, wherein the nanoparticles comprise dextran nanoparticles.

8. The composition of claim 1, wherein the hydrogel is a thermosensitive hydrogel, or an injectable hydrogel.

9. The composition of claim 8, wherein the thermosensitive hydrogel comprises a polyoxyethylene-polyoxypropylene block copolymer, and the composition is in the form of a hydrogel patch.

10. The composition of claim 8, wherein the injectable hydrogel comprises heparan, heparan sulfate, chitosan, hyaluronic acid, dextran, alginic acid, hydroxyethyl starch, or a combination thereof, and a reactive crosslinker, and the composition is in the form of an injectable hydrogel.

11. The composition of claim 1, further comprising an immune checkpoint inhibitor, such as a peptide with high affinity for an immune checkpoint receptor, wherein the immune checkpoint receptor comprises PD-L1, PD-1, OX40, TIGIT, CTLA-4, CD137 (4-1BB), CD28, and CD27.

12. A method of treating a primary or metastatic tumor, comprising locally injecting the composition of claim 10 at the site of the primary or metastatic tumor.

13. The method of claim 12, wherein the metastatic tumor is a metastatic breast cancer tumor.

14. The method of claim 12, wherein the primary tumor is a melanoma, a sarcoma, prostate cancer, or cervical cancer.

15. The method of claim 12, wherein the composition further comprises an immune checkpoint inhibitor, such as a peptide with high affinity for an immune checkpoint receptor, wherein the immune checkpoint receptor comprises PD-L1, PD-1, OX40, TIGIT, CTLA-4, CD137 (4-1BB), CD28, and CD27.

16. A method of treating an inoperable cancer, comprising implanting the composition of claim 9 at the site of the inoperable cancer.

17. The method of claim 16, wherein the inoperable cancer is inoperable ovarian cancer, inoperable lung cancer, inoperable pancreatic cancer, inoperable liver cancer, or inoperable colorectal cancer.

18. The method of claim 16, wherein the composition further comprises an immune checkpoint inhibitor, such as a peptide with high affinity for an immune checkpoint receptor, wherein the immune checkpoint receptor comprises PD-L1, PD-1, OX40, TIGIT, CTLA-4, CD137 (4-1BB), CD28, and CD27.


SEQUENCE LISTING (2 sequences)

SEQ ID NO: 1 - protein, 325 aa, Mus muscalis
MPSAFEKVVKNVIKEVSGSRGDLIPVDSLRNSTSFRPYCLLNRKFSSSRFWKPRYSCVNLSIKDILEPSAPEPEPECFGSFKVSDVVDGNIQGRVMLSGMGEGKISGGAAVSDSSSASMNVCILRVTQKTWETMQHERHLQQPENKILQQLRSRGDDLFVVTEVLQTKEEVQITEVHSQEGSGQFTLPGALCLKGEGKGHQSRKKMVTIPAGSILAFRVAQLLIGSKWDILLVSDEKQRTFEPSSGDRKAVGQRHHGLNVLAALCSIGKQLSLLSDGIDEEELIEAADFQGLYAEVKACSSELESLEMELRQQILVNIGKILQDQ

SEQ ID NO: 2 - protein, 484 aa, Homo sapiens
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH